(19) United States
(12) Patent Application Publication
(10) Pub. No.: US 2005/0027316 A1
(43) Pub. Date: Feb. 3, 2005

(54) Resurrection burial tomb

(76) Inventor: Daniel Robert Izzo, Syracuse, NY (US)

Correspondence Address: Reverend Daniel Robert Izzo, 512 Onondaga Ave, Syracuse, NY, 13207, US

(21) Appl. No.: 10/161,974

(22) Filed: Jun. 4, 2002

Related U.S. Application Data
(63) Continuation-in-part of application No. 09/619,698, filed on Jul. 19, 2000, now abandoned.
(63) Continuation-in-part of application No. 09/205,415, filed on Dec. 2, 1998, now abandoned.
Application No. 09/205,415 is a continuation-in-part of application No. 08/798,316, filed on Feb. 10, 1997, now abandoned.
Application No. 09/619,698 is a continuation-in-part of application No. 09/177,760, filed on Oct. 24, 1998, now abandoned.
Application No. 09/177,760 is a continuation-in-part of application No. 08/813,825, filed on Aug. 21, 1997, now abandoned.
Application No. 09/177,760 is a continuation-in-part of application No. 09/239,269, filed on Jan. 26, 1999, now abandoned.
Application No. 09/619,698 is a continuation-in-part of application No. 08/766,130, filed on Dec. 16, 1996, now Pat. No. 6,064,047.
(60) Provisional application No. 60/295,356, filed on Jun. 4, 2001.
(60) Provisional application No. 60/144,245, filed on Jul. 19, 1999.
(60) Provisional application No. 60/063,068, filed on Oct. 27, 1997.
(60) Provisional application No. 60/072,624, filed on Jan. 26, 1998.
(60) Provisional application No. 60/148,654, filed on Aug. 13, 1999.
(60) Provisional application No. 60/072,579, filed on Jan. 26, 1998.

(51) Int. Cl. A61N 1/00

(52) U.S. Cl. 607/1

(57) ABSTRACT

A Resurrection Burial Tomb includes a means to preserve and revive Human Beings and provide power and power systems for the same, comprising of a container of preservation means, holding the suspended dead person's body and connected to electrical and energy apparatus systems contained in the Resurrection tomb and robotic machine workers that help maintain and work to revive the suspended Huamn Being; wherein the process provides power and security from death to living Human Beings being useful and novel, producing a less savage empowered child culture and machine parents.

Application Ser. No. 10/161,974

A RESURRECTION BURIAL TOMB in this application is a CIP of application Ser. No. 09/619,698 filed Jul. 19, 2000, which is a CIP of “A Resurrection Machine and Consciousness Revival System” application No. 60/144,245 filed Jul. 19, 1999 and a CIP of “The Thermionic Electron Electric Generator and Artifical Liver Ammonia Diffuser” application No. 60/295,356; filed on Jun. 04, 2001 and “The Microwave Hot Water Boiler Heating System” application Ser. No. 08/766,130 filed Dec. 16, 1996 U.S. Pat. No. 6,064,047 with Discloser Document #412019 priority, filed Jan. 10, 1997 and “The Portable Nuclear Powered Engine and Electric Generator” application Ser. No. 09/205,415 filed Dec. 02, 1998 (being a CIP of application Ser. No. 08/798,316 filed on Feb. 10, 1997) and “The Hydraulic Leverage Engine” application Ser. No. 09/177,760 filed on Oct. 24, 1998 (being a CIP of application Ser. No. 08/813,825 filed on Aug. 21, 1997 and application No. 60/063,068 filed on Oct. 27, 1997 and application No. 60/072,624 filed on Jan. 26, 1998 and “The Zinc-Carbon Pill and Transistorized Plastic Paper Computer” application Ser. No. 09/239,269 filed on Jan. 26, 1999 (being a CIP of application No. 60/072,579 filed on Jan. 26, 1998) and “The Artifical Muscle Robot” application No. 60/148,654 filed on Aug. 13, 1999 Pat. App No. 60/128,747 filed on Apr. 12, 1999 “Free Citizen's Band Public Radiowave Telephone System and Computer” Together with priority “conception of invention claims ” of “The Artifical Heart Assistor Pump” application No. 60/122,189 filed on Mar. 01, 1999 and Disclosure Document #452294 filed on Mar. 03, 1999 Together with priority “conception of invention claims” of: Disclosure Document #412019 filed on Jan. 10, 1997; Discloser Document #452294; Discloser Document #494846; Discloser Document #486705; Discloser Document #494219 filed May 21, 2001; Discloser Document #493,675 filed May 17, 2001; Discloser Document #493701;

The above references are held the priority and inventions, of the applicant, the Reverend Daniel Robert Izzo of 512 Onondaga Ave., Syracuse New Your 13207 citizen of the United States of America, who has not abandoned any of his science inventions, obviously, but views them as a form of his children.

### BACKGROUND OF THE INVENTION

1. Field of the Invention

This invention pertains to processes and devices that provide power and security from death, and more particularly, a noble branch or field thereof; known in psychoanalysis as an empowerment means that relieves some fears of eternal death and the anxiety about the same, together with devices to empower people who lack independent power sources.

2. Background Information

In order to provide background information so that the invention maybe completely understood and appreciated in its proper context, reference may be made to a number of prior art patent and applications of the inventor as follows.

### SUMMARY OF THE INVENTION

It is another princple object of the invention; to provide the present Human Community, being sadomasocistic by nature; (re; cruel world, death and taxes; master-slave; cop-robber; landlord-tenant; schoolmaster-pupil; boss-worker etc.); with a more fun and better, self supporting prolonged Child Culture having Robot Parents and energy sources to empower the same; (re; man and machine), the inventor believes that this is the next progression in Human development, wherein goverment makes its own tax es from gold coins transmutated from neutron bombarded lead and engineers a sort of made-made heaven; wherein the work is play and fun, inpart. Psychoanalysis shows that the 3 main issues that human beings deal with are issues of 1) Power, (food, heat, ect.) 2) Sex Gratification, (creation of children) 3) Security from Death. (religion tales anxiety relief, peace ect.) because they have survival value. This invention attempt provide human beings with issues; (1) Power; and a little more of issue (3) Security from Death.

### BRIEF DESCRIPTION OF THE DRAWINGS

FIG. 1

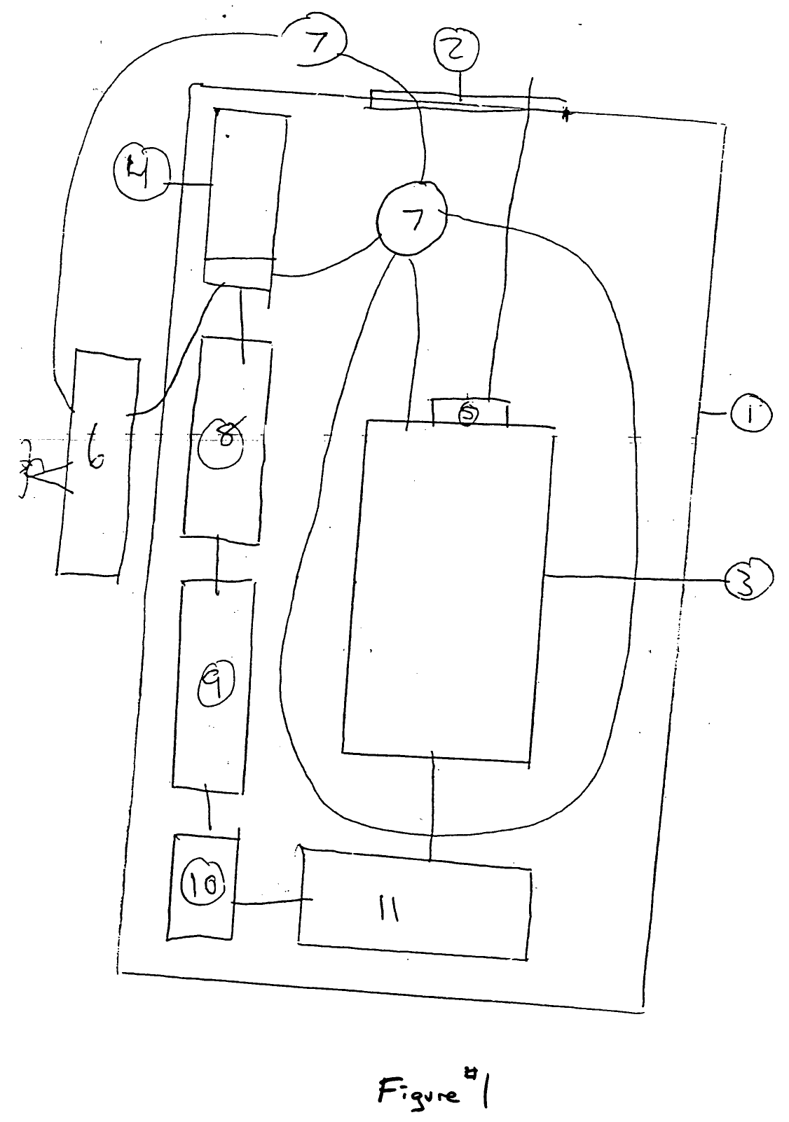

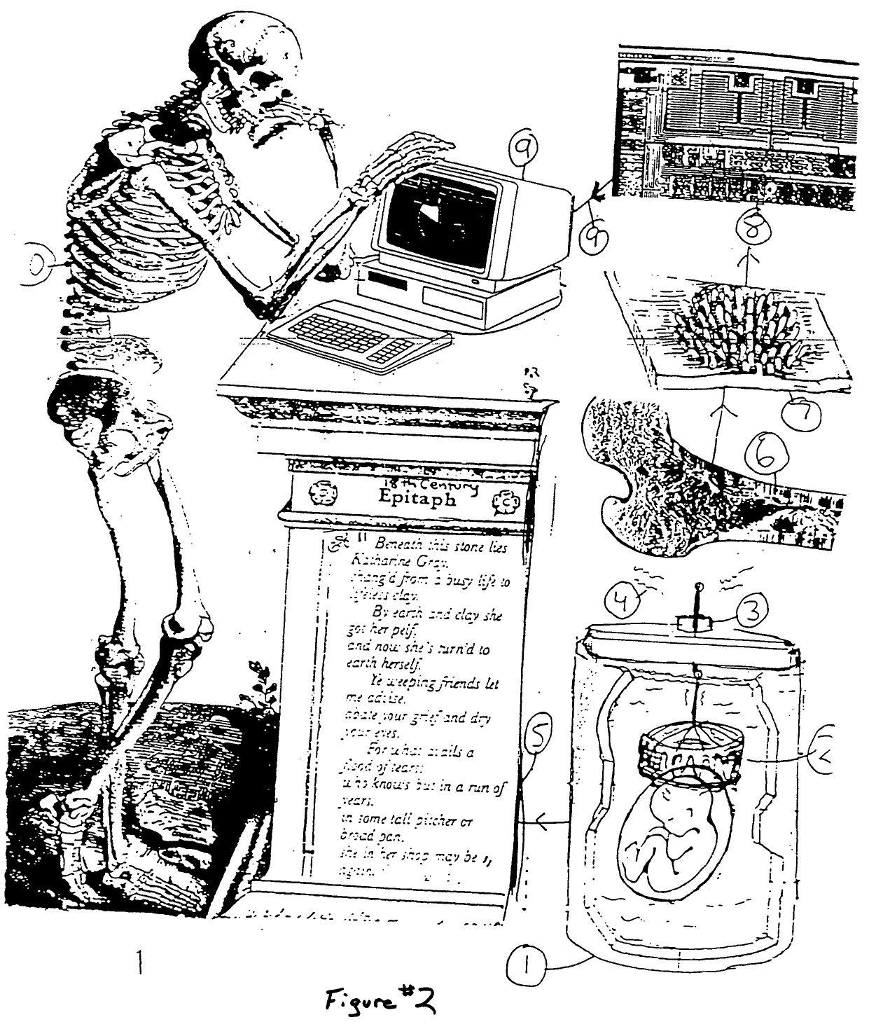

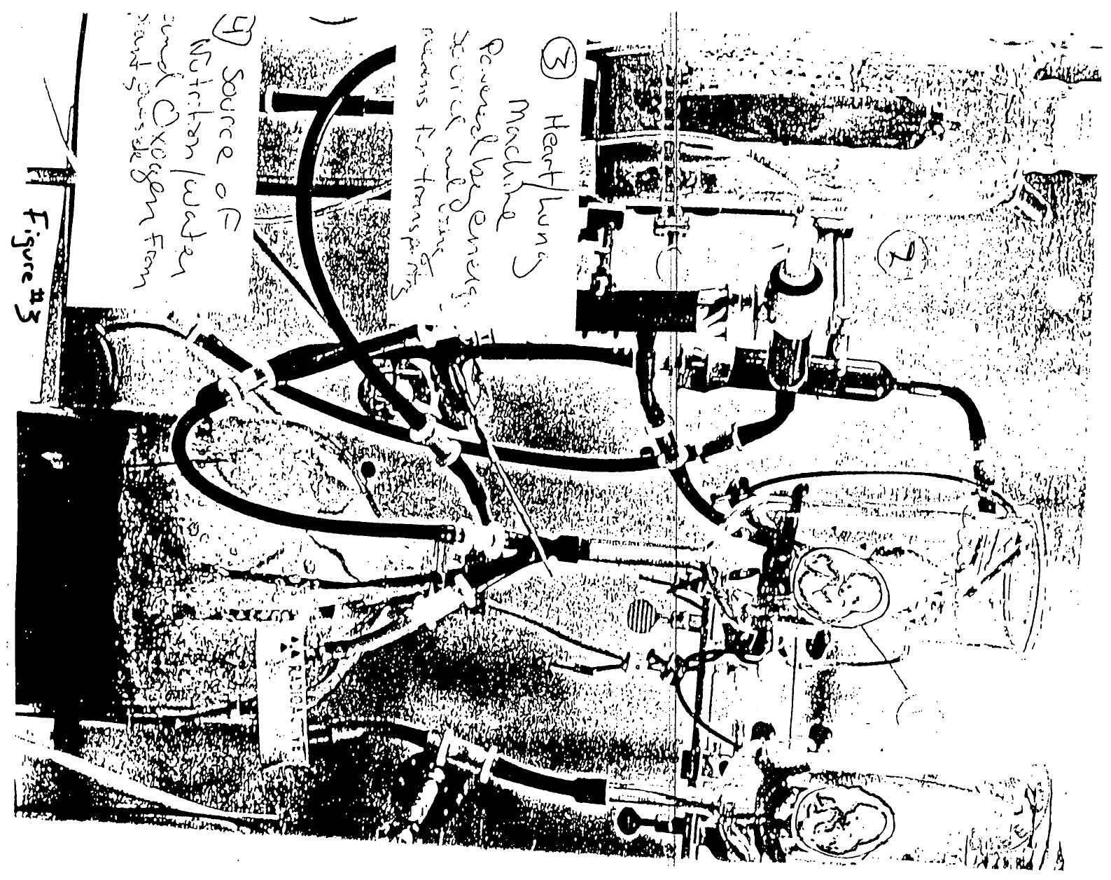

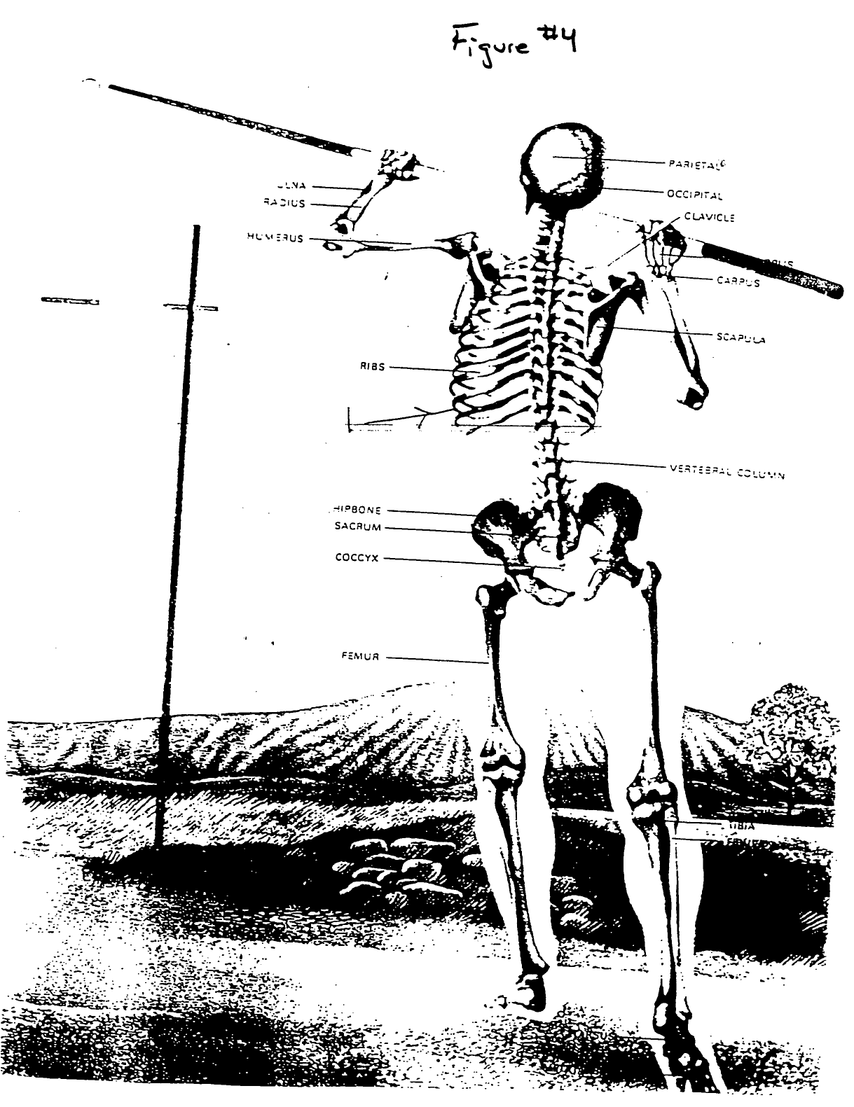

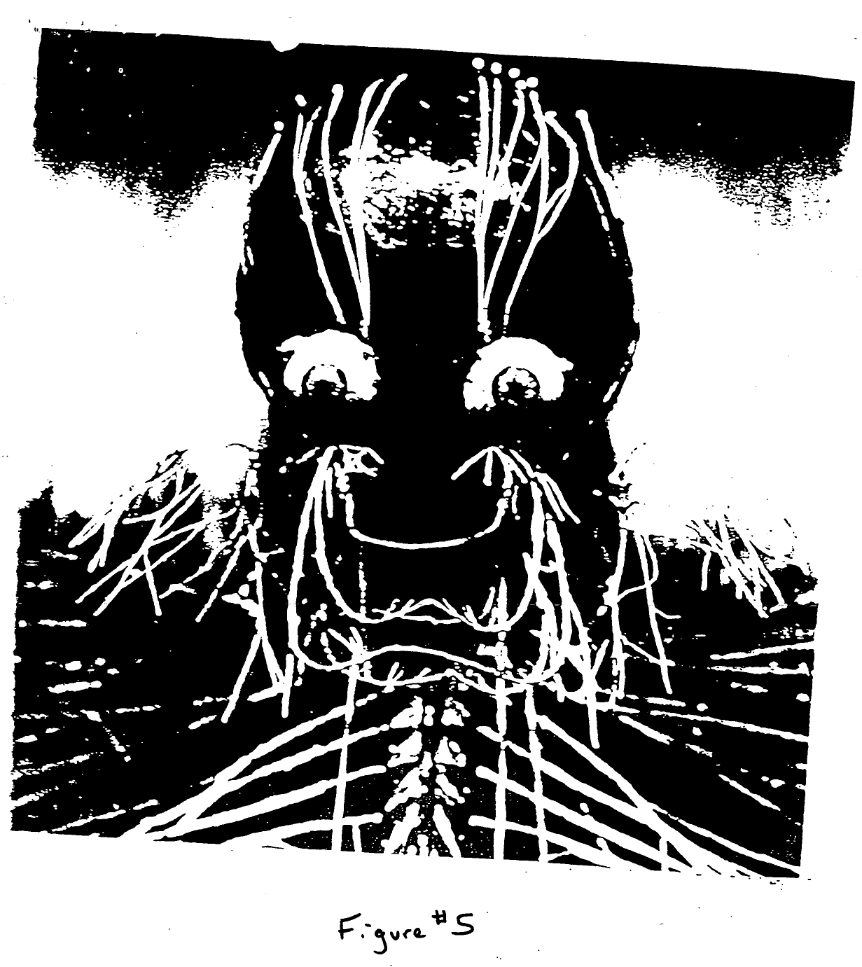

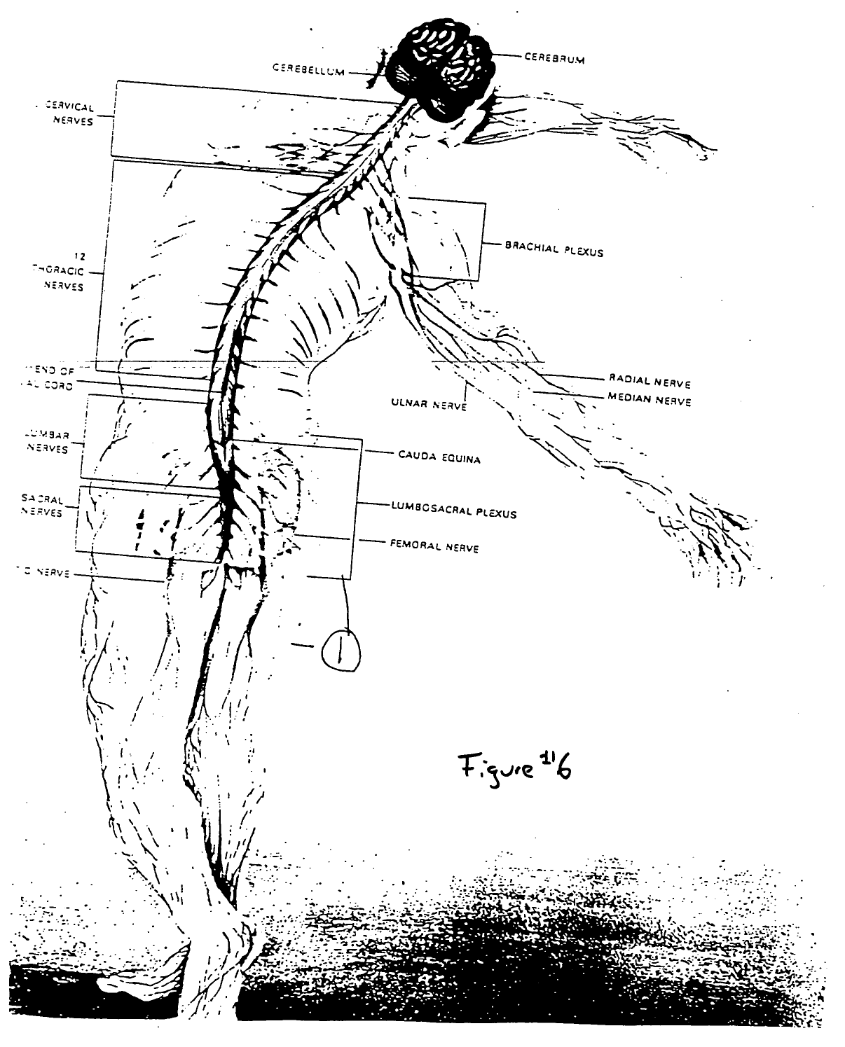

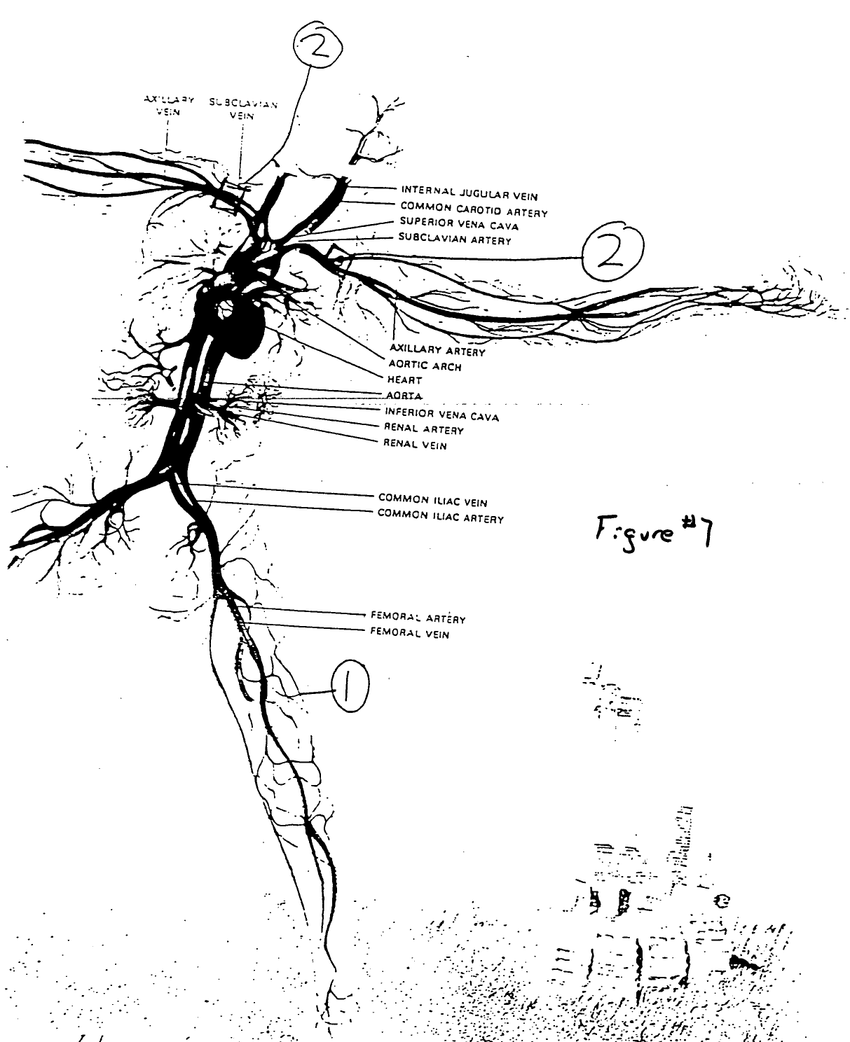

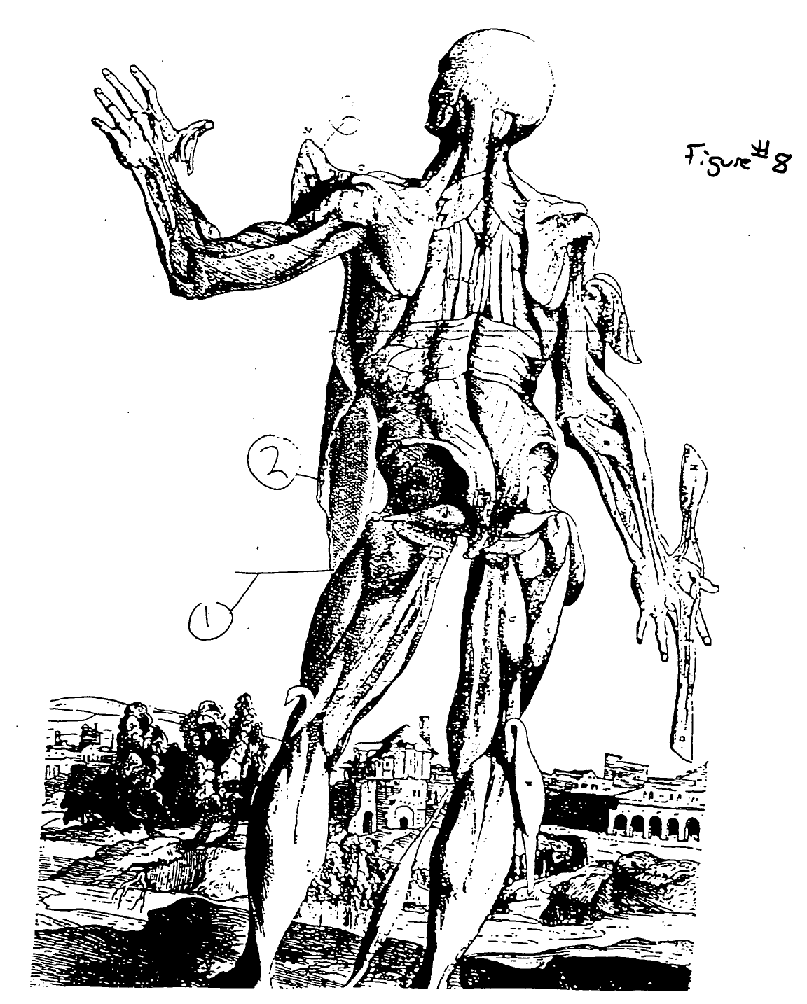

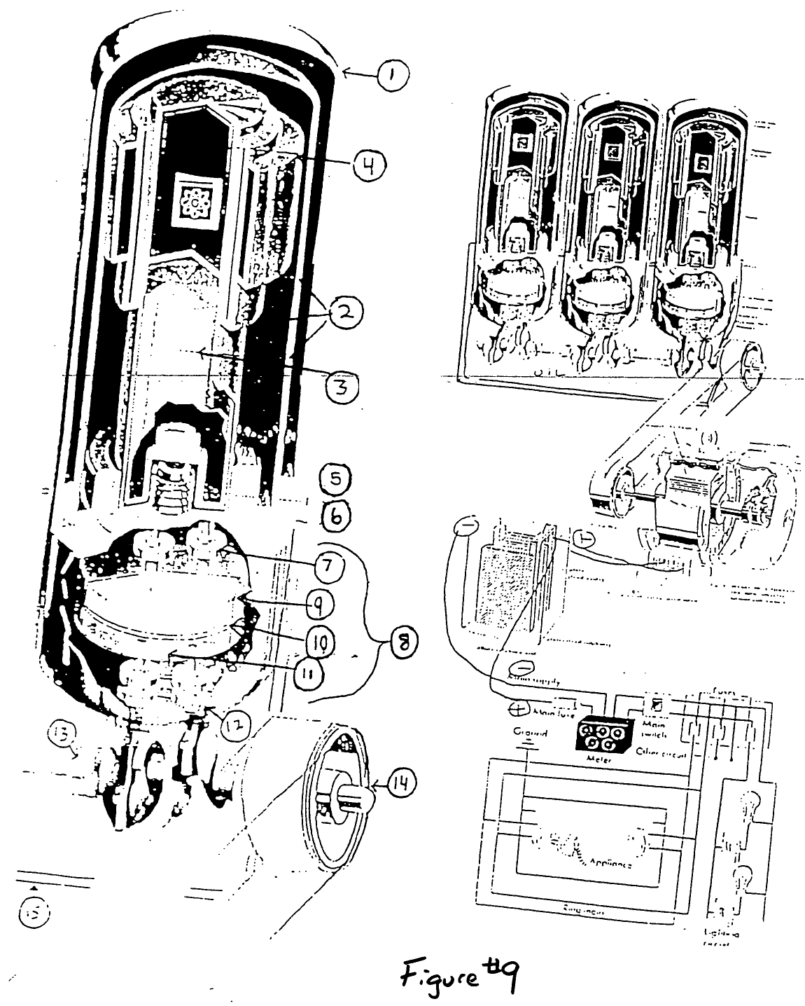

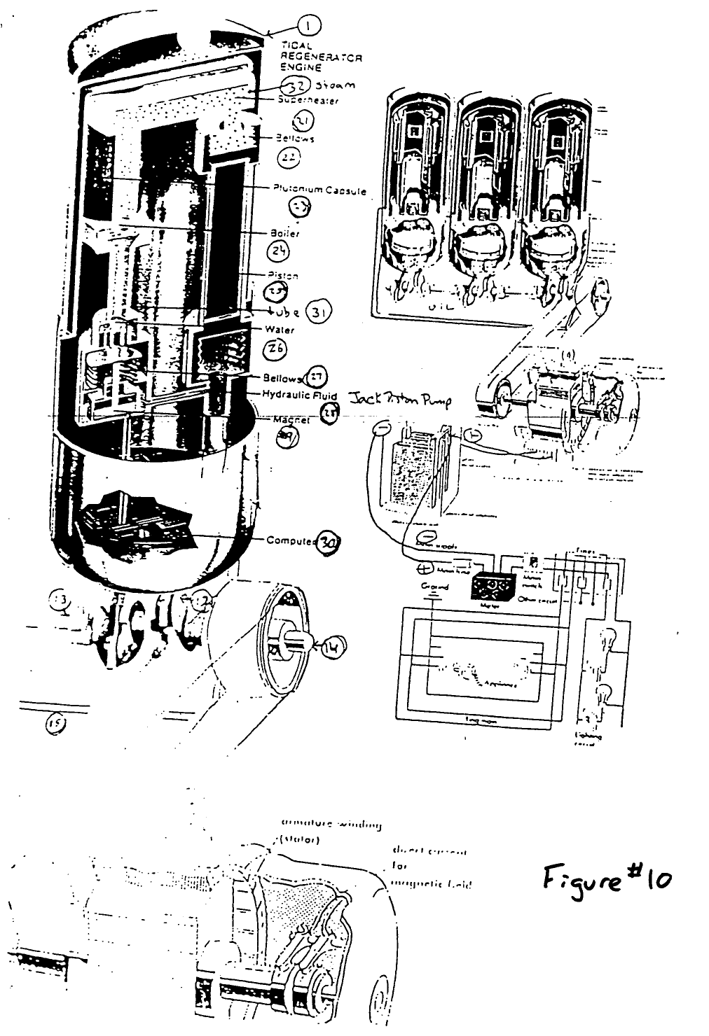

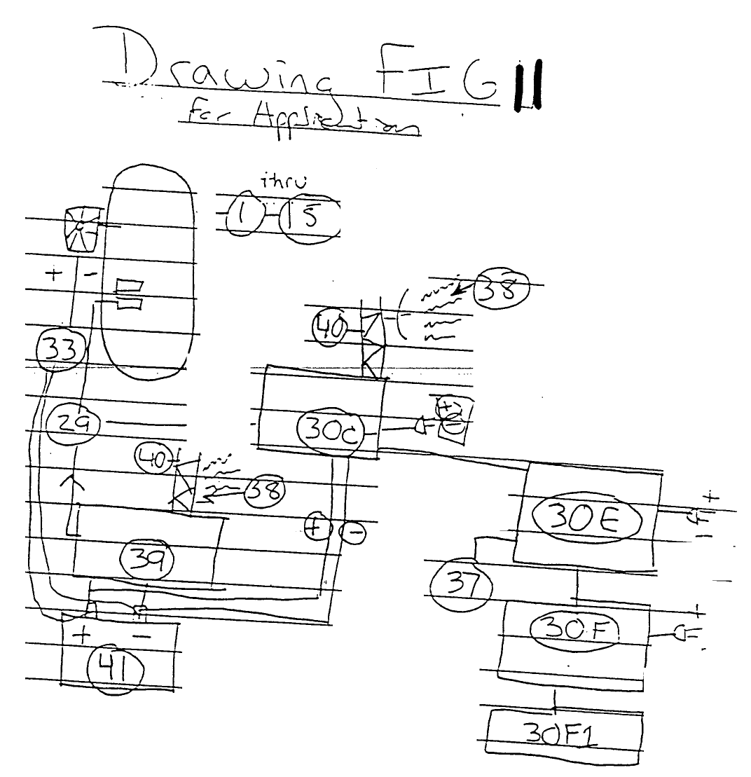

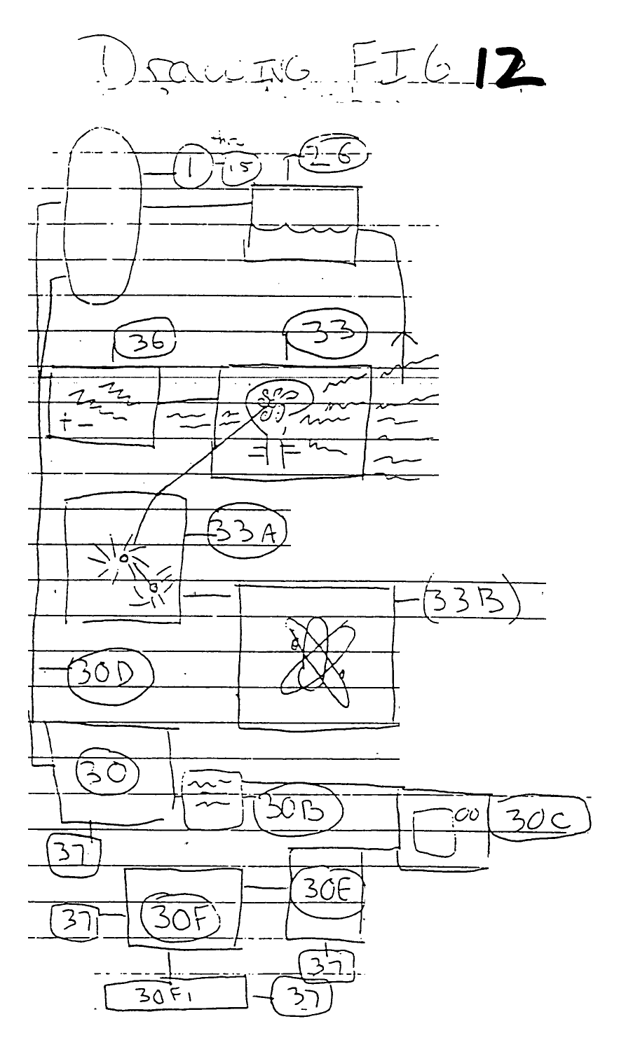

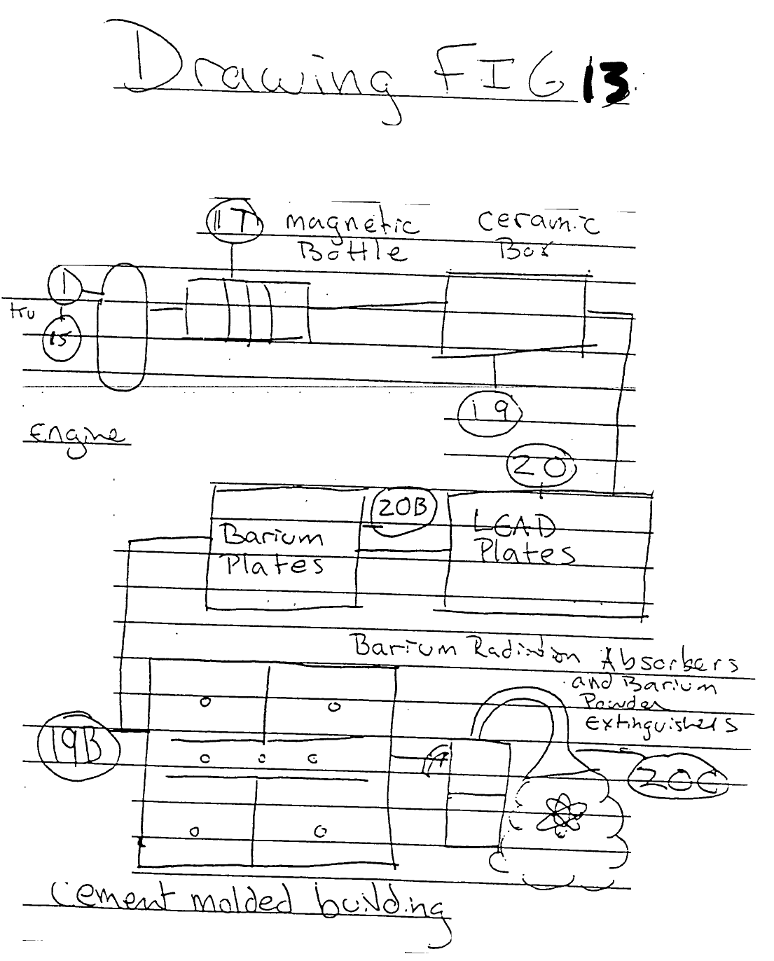

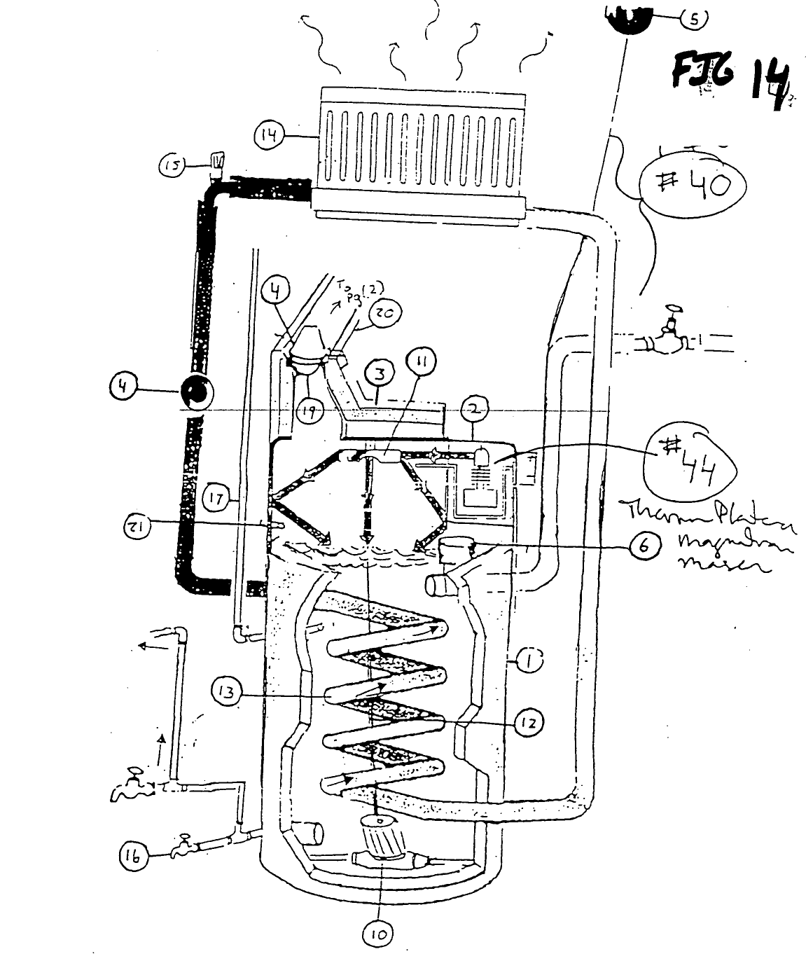

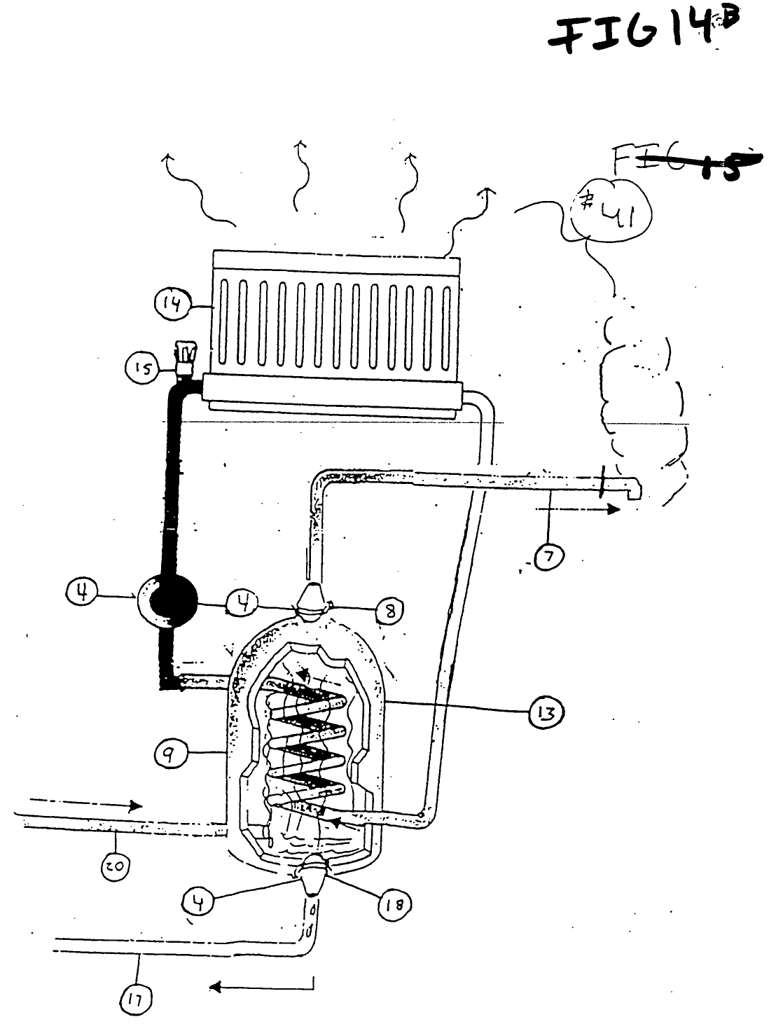

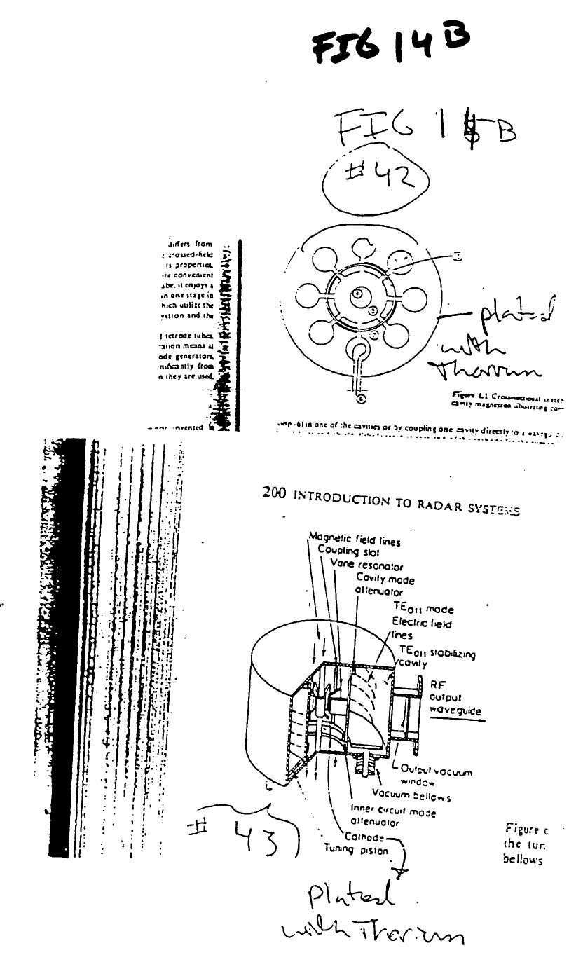

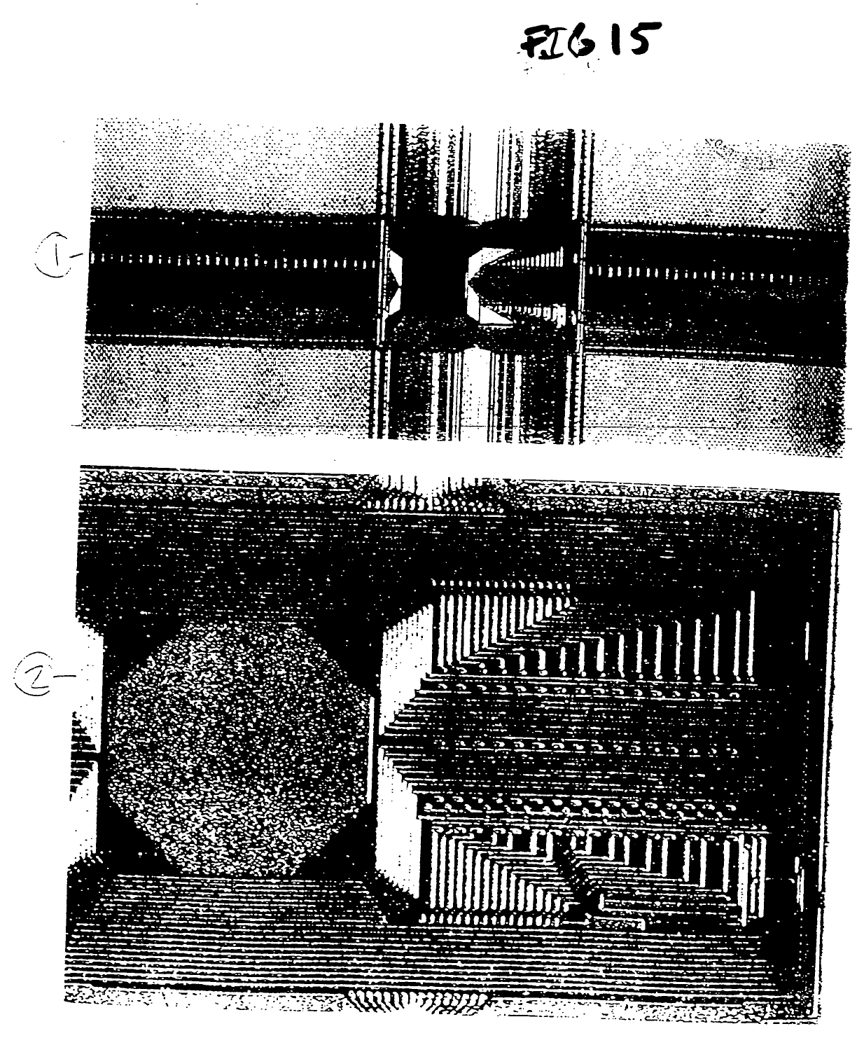

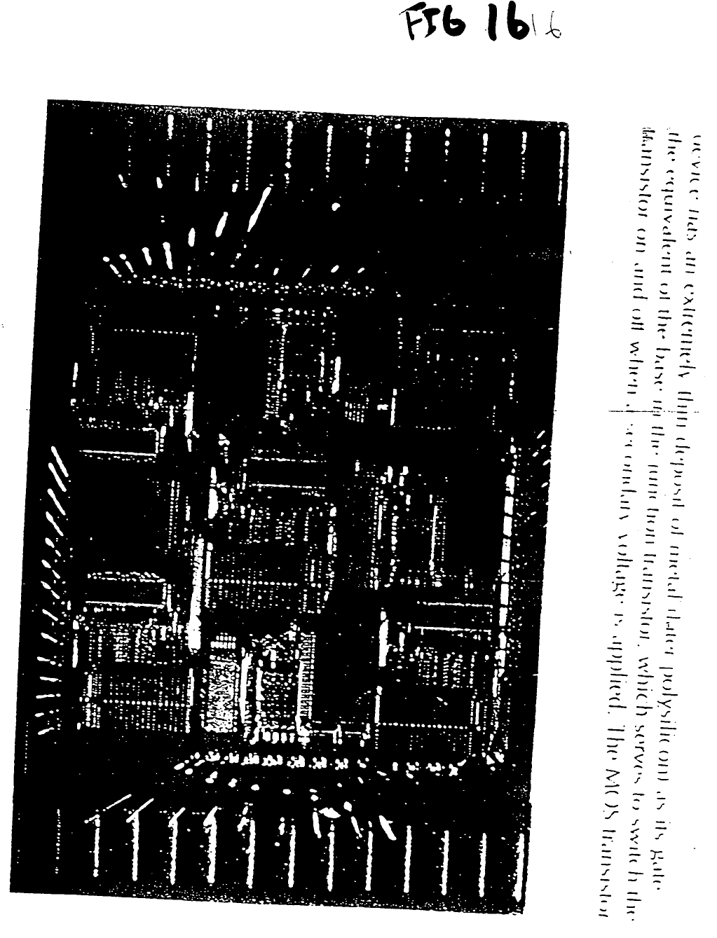

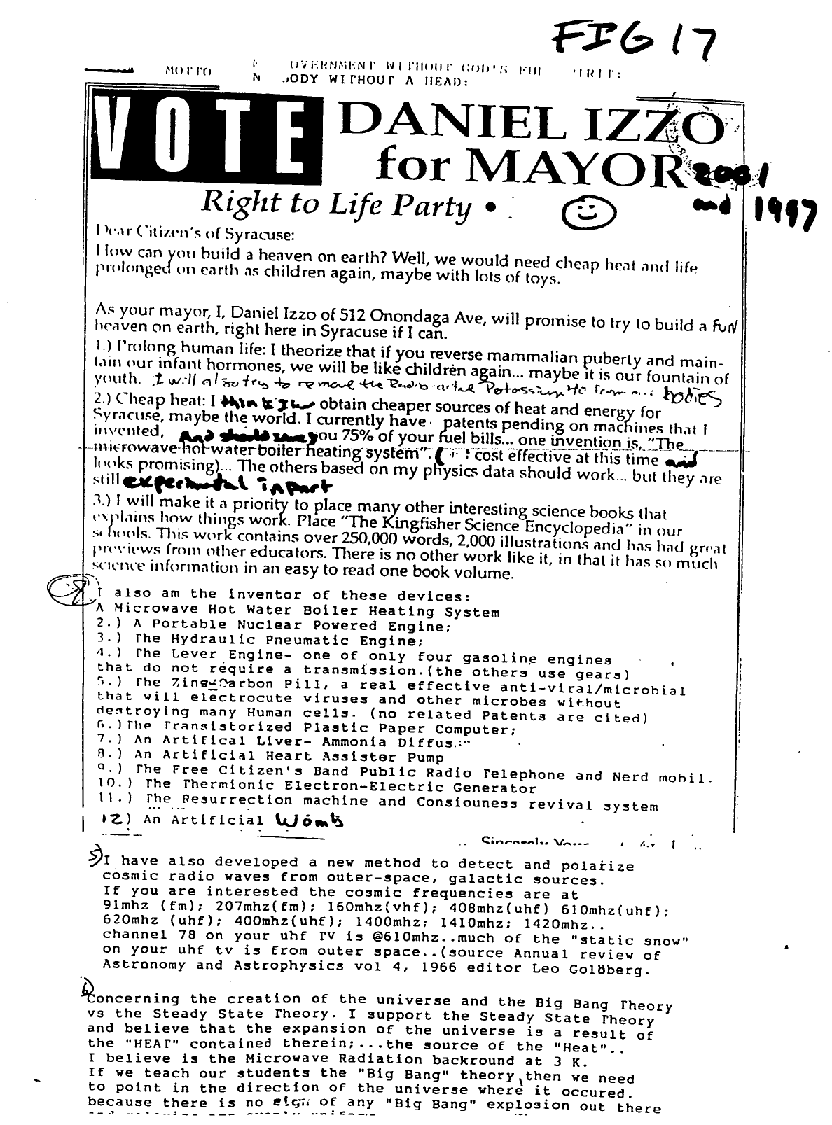

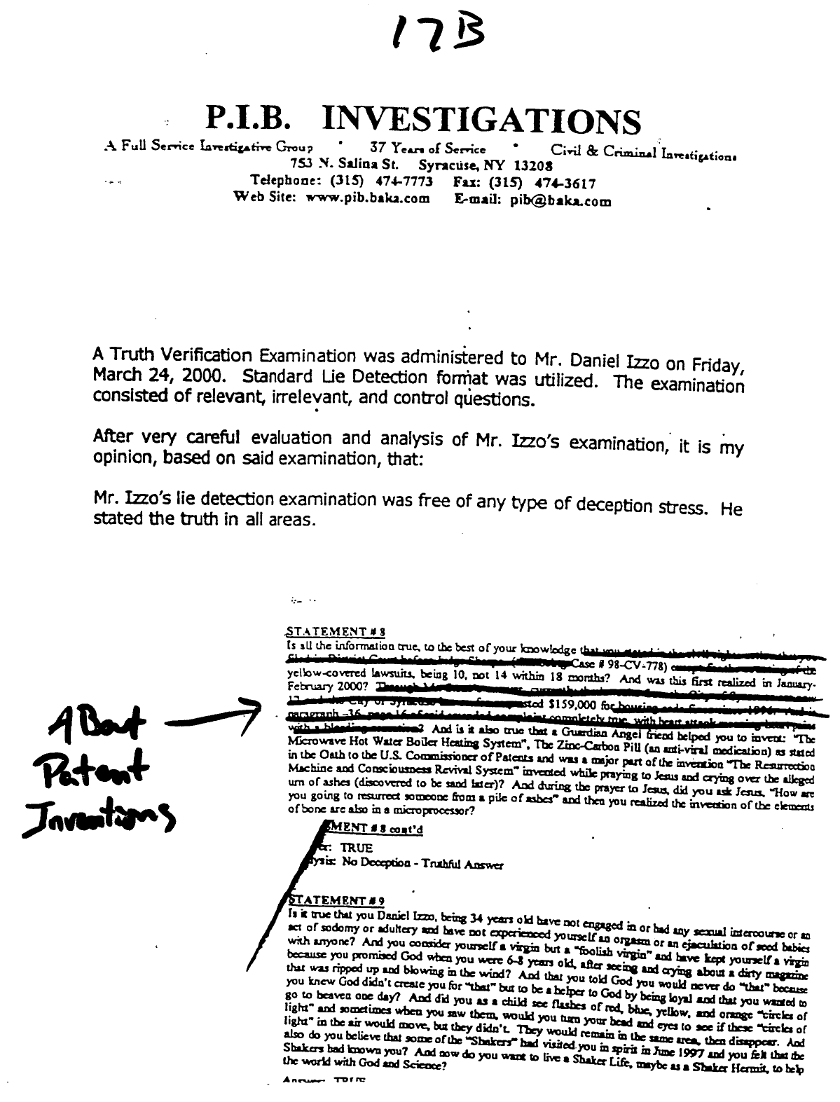

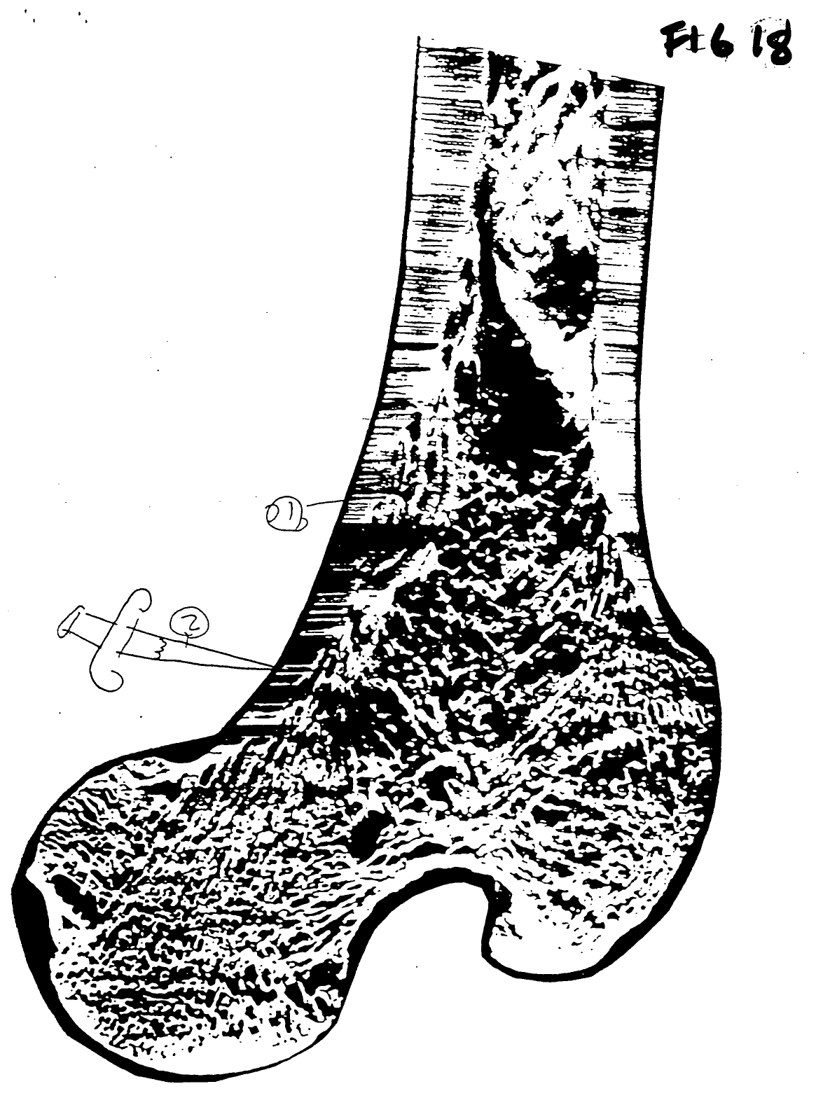

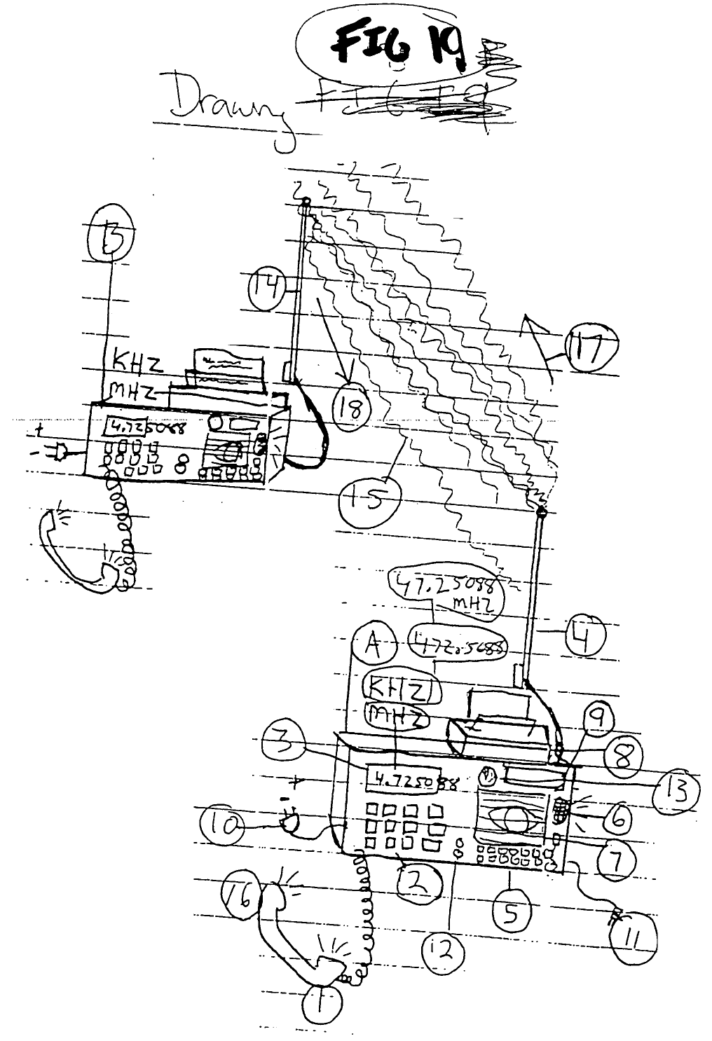

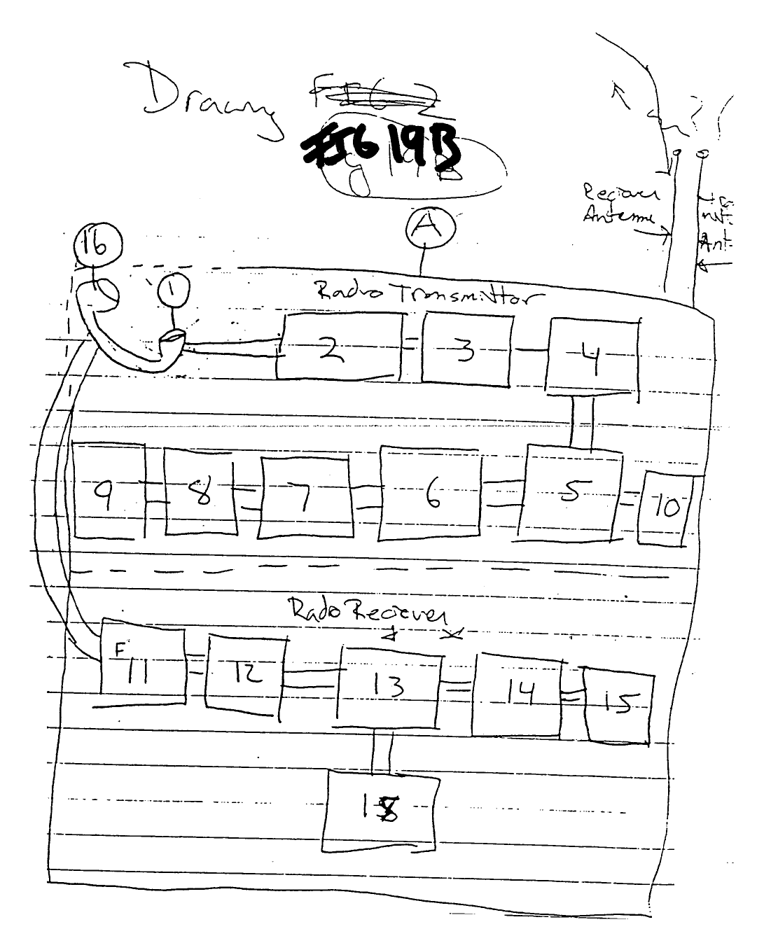

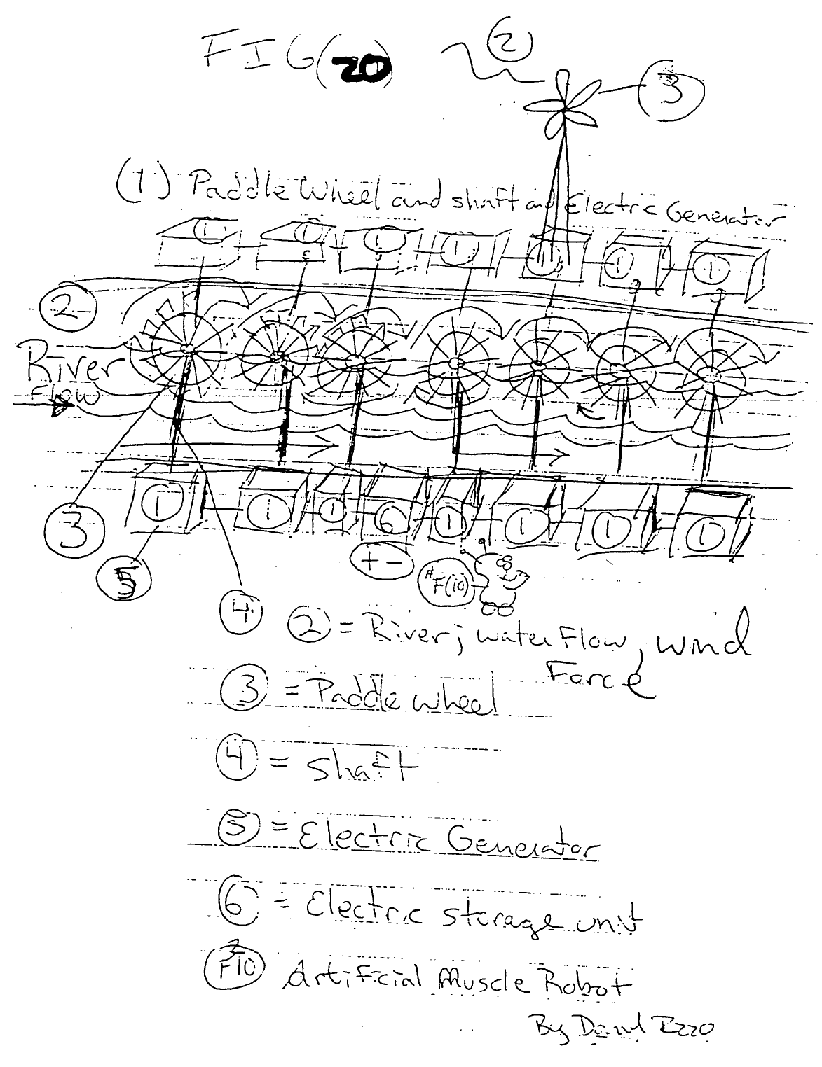

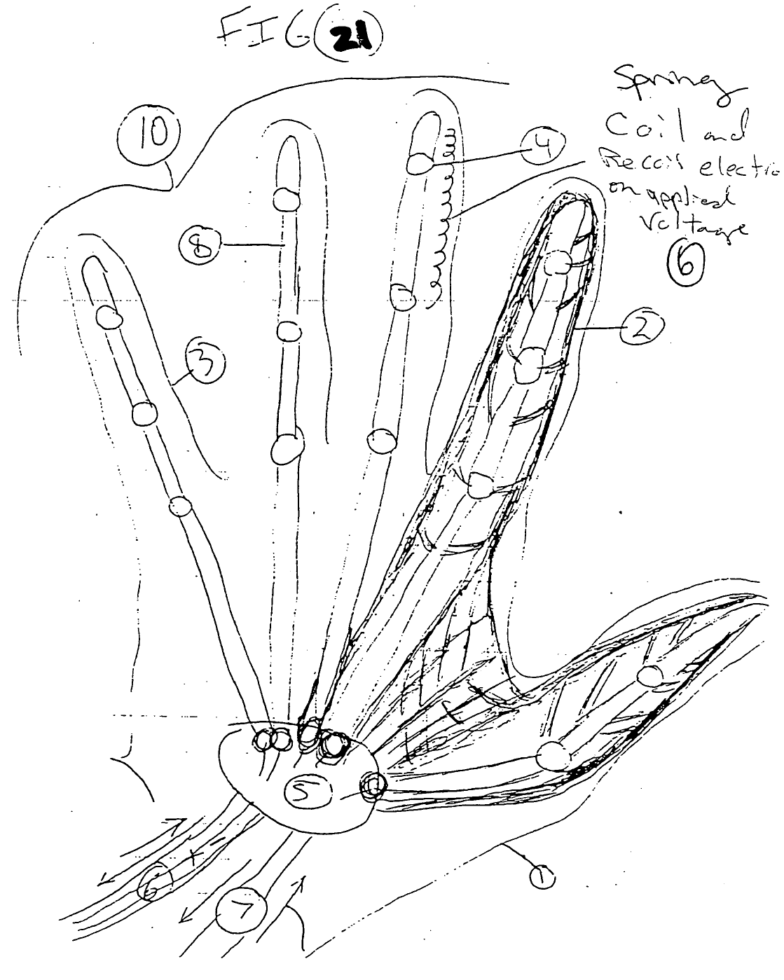

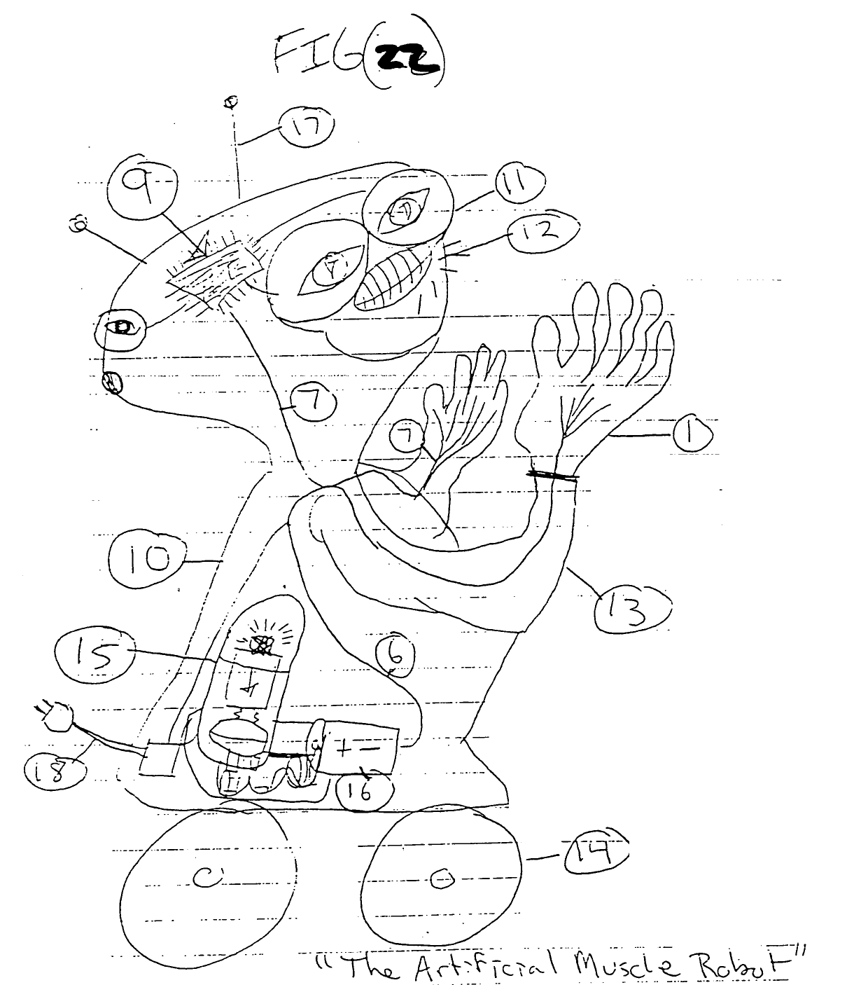

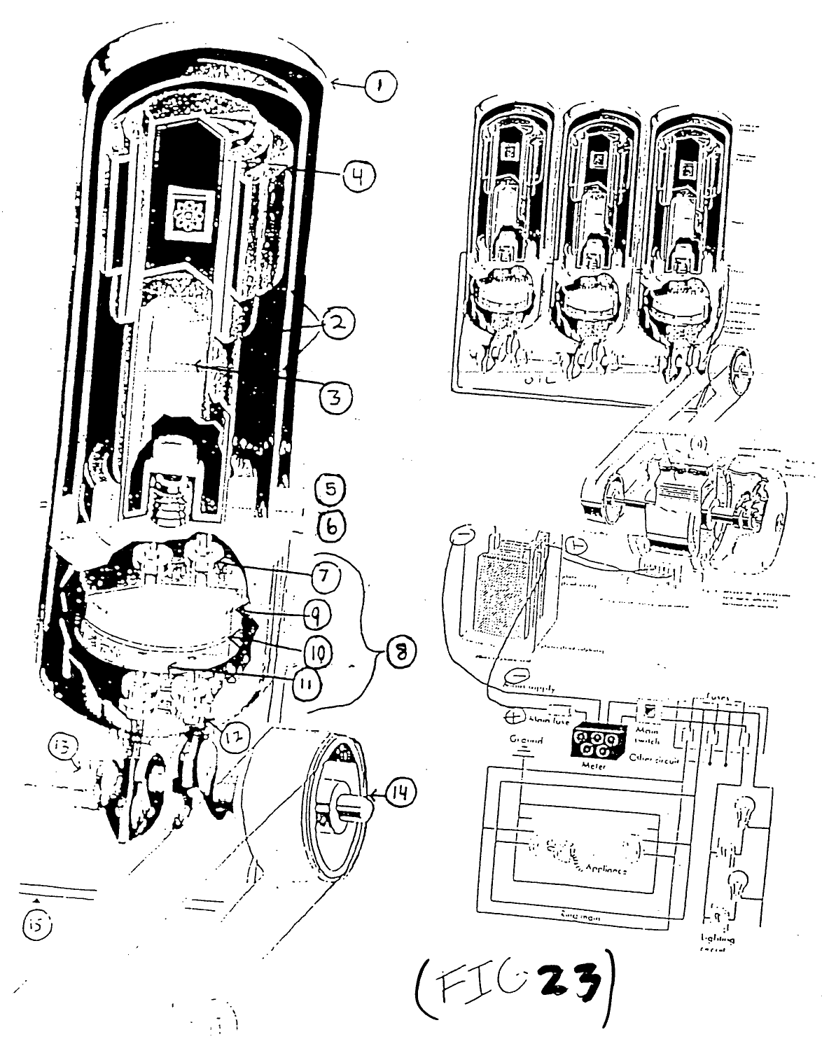

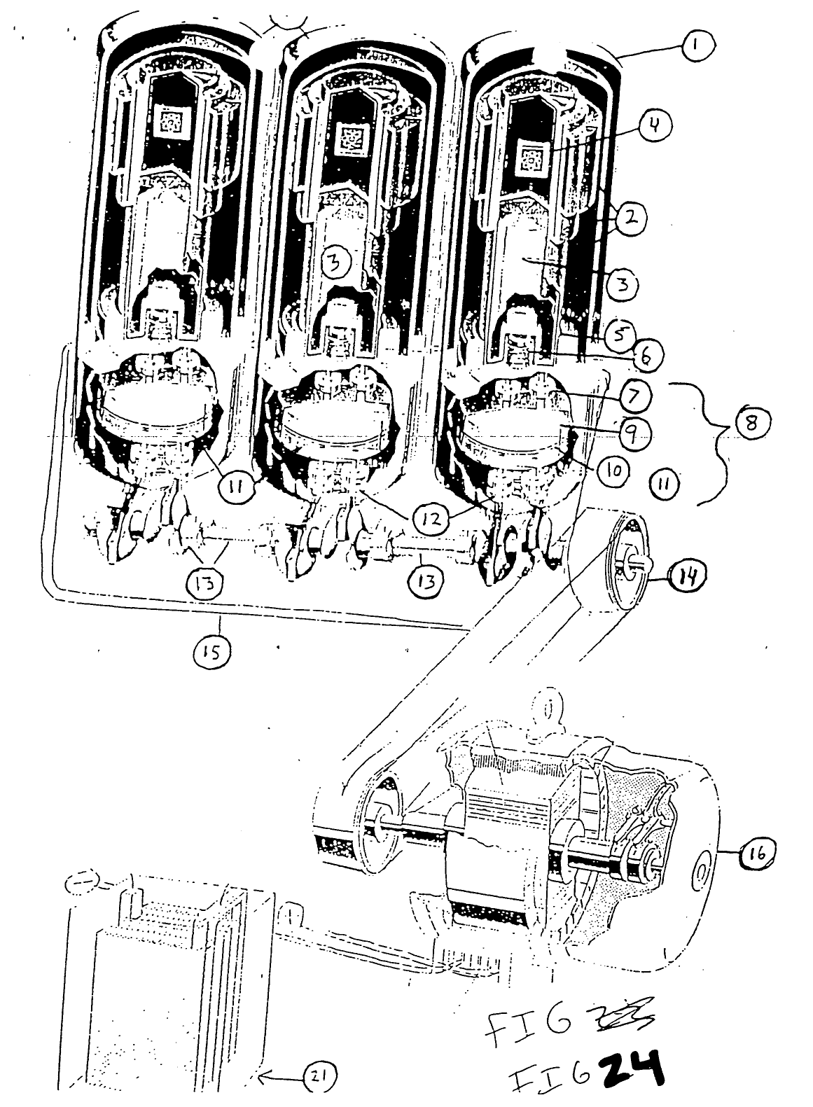

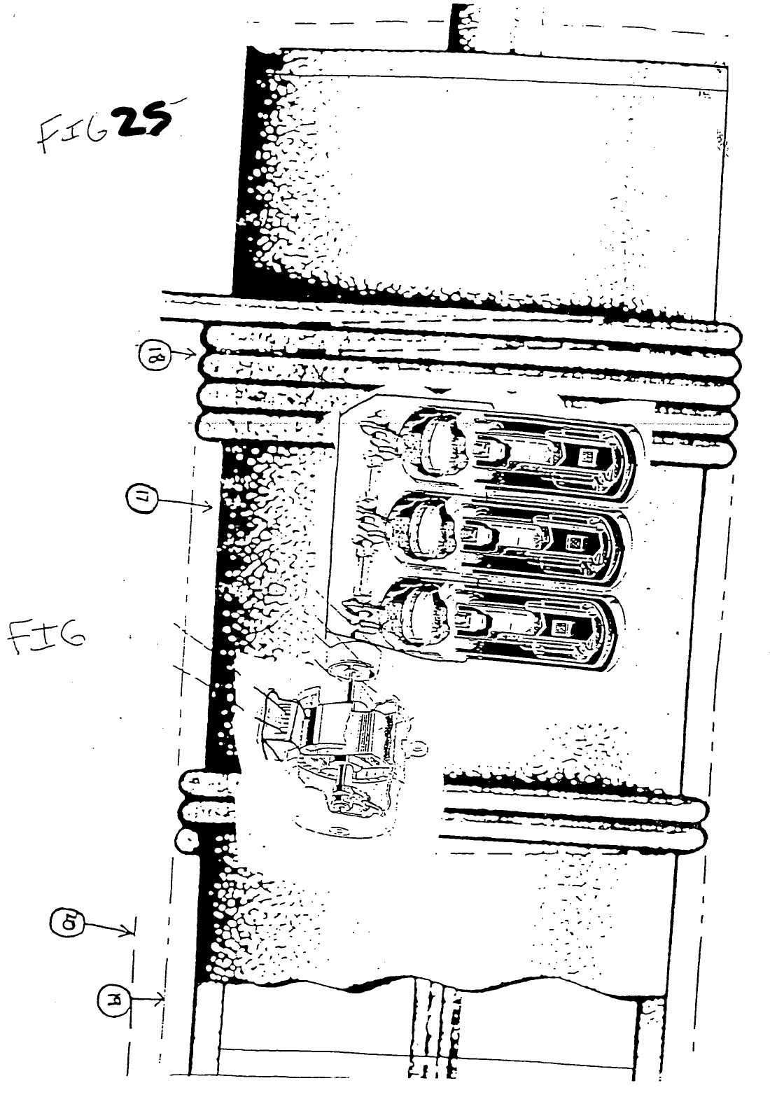

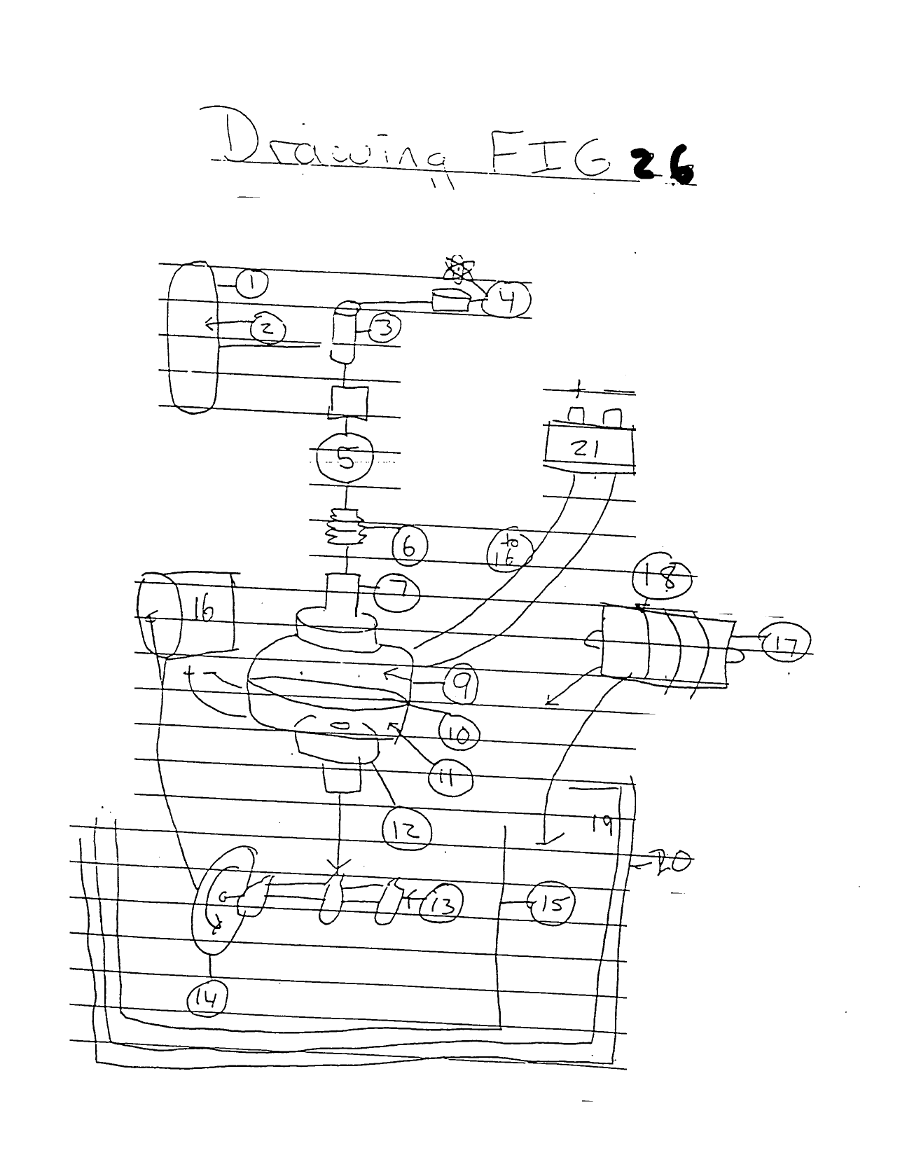

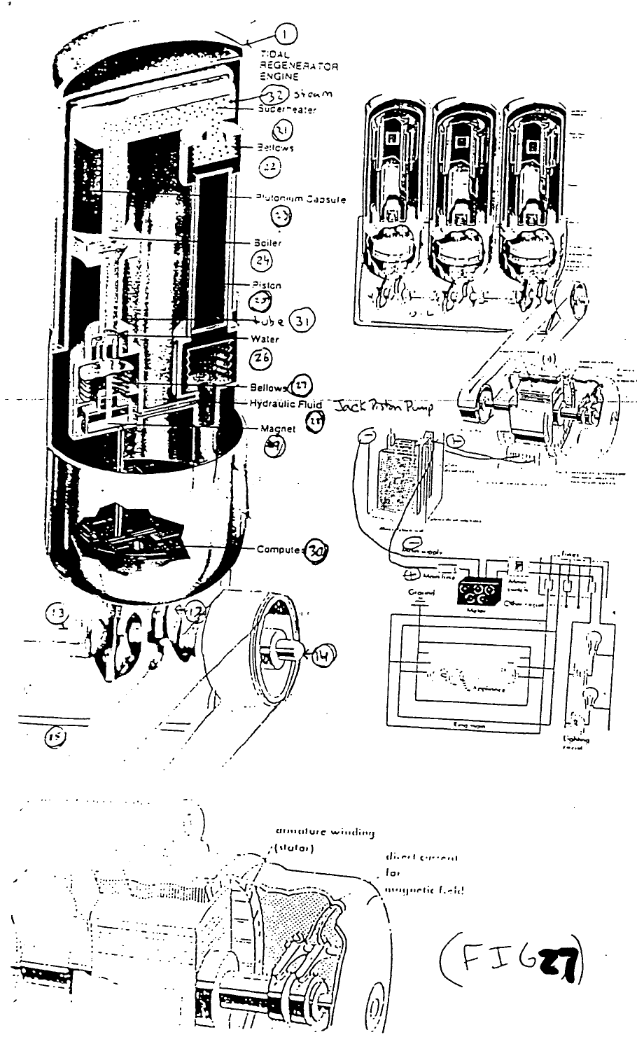

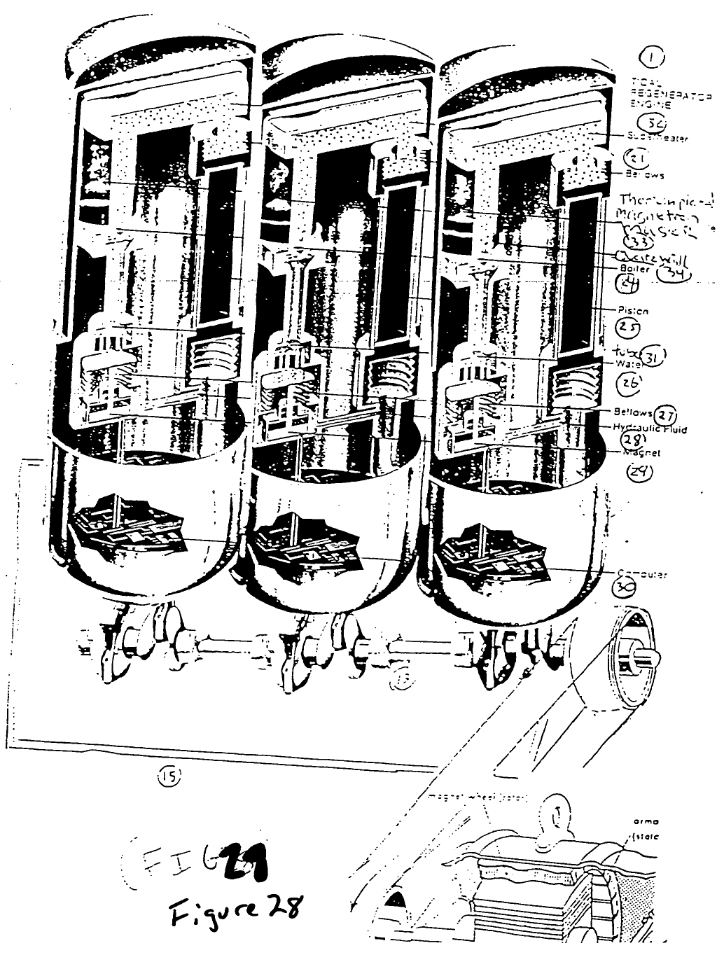

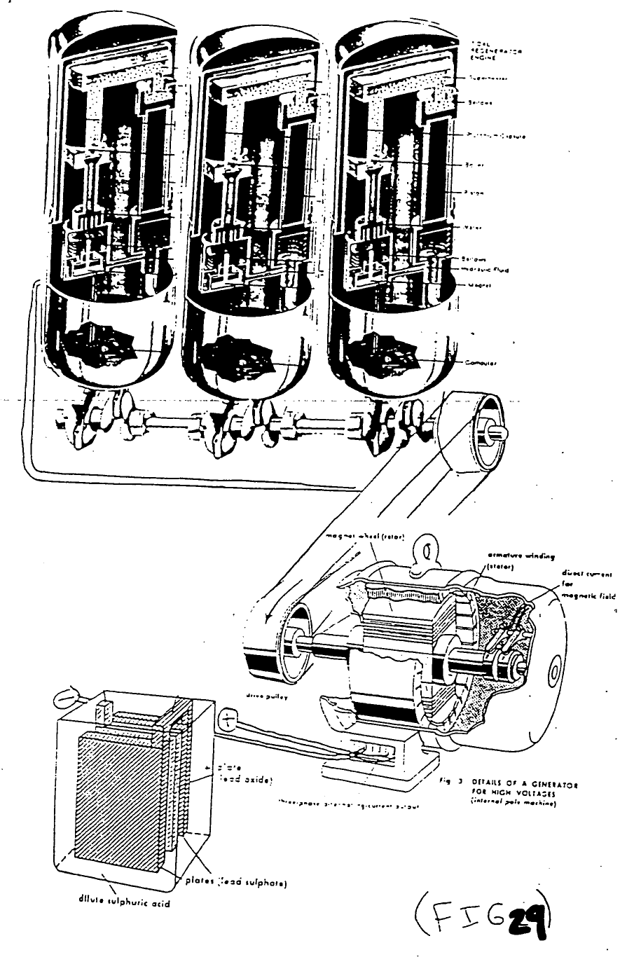

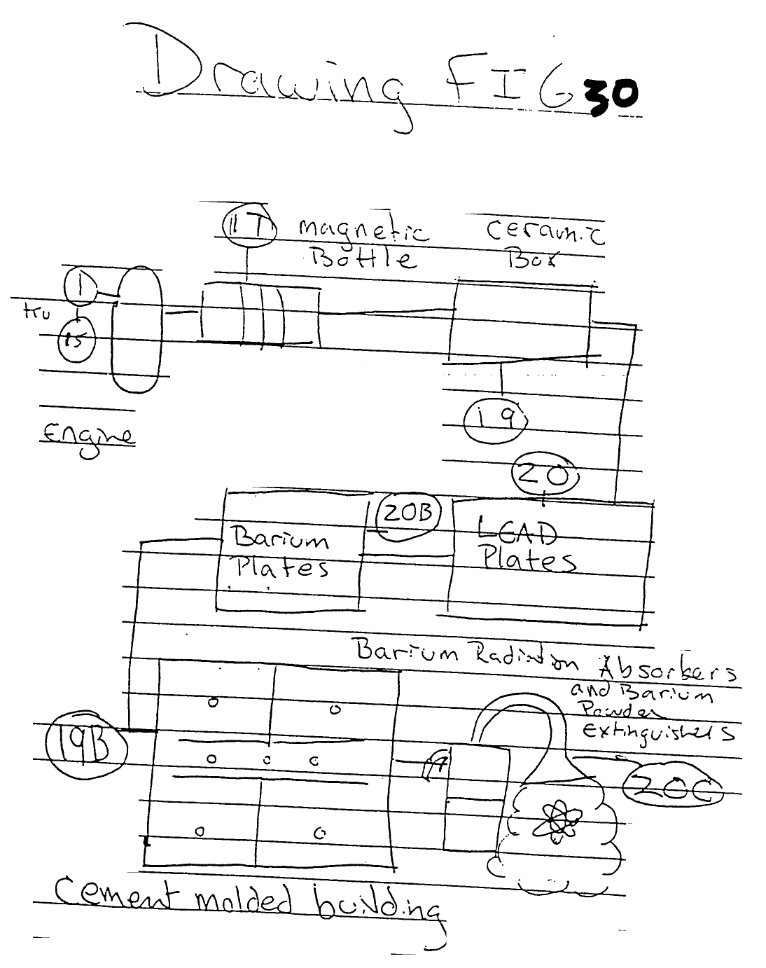

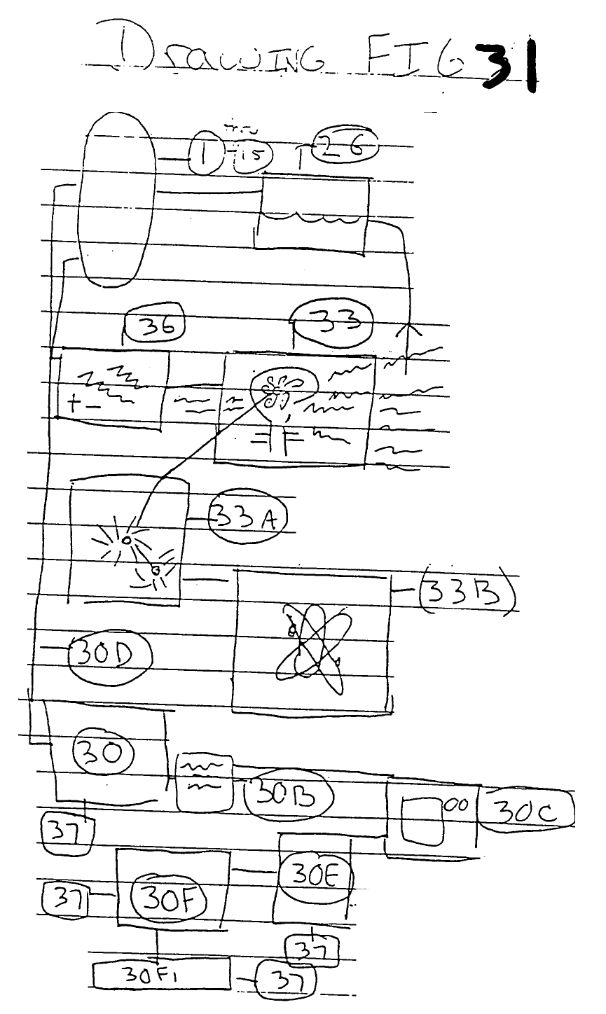

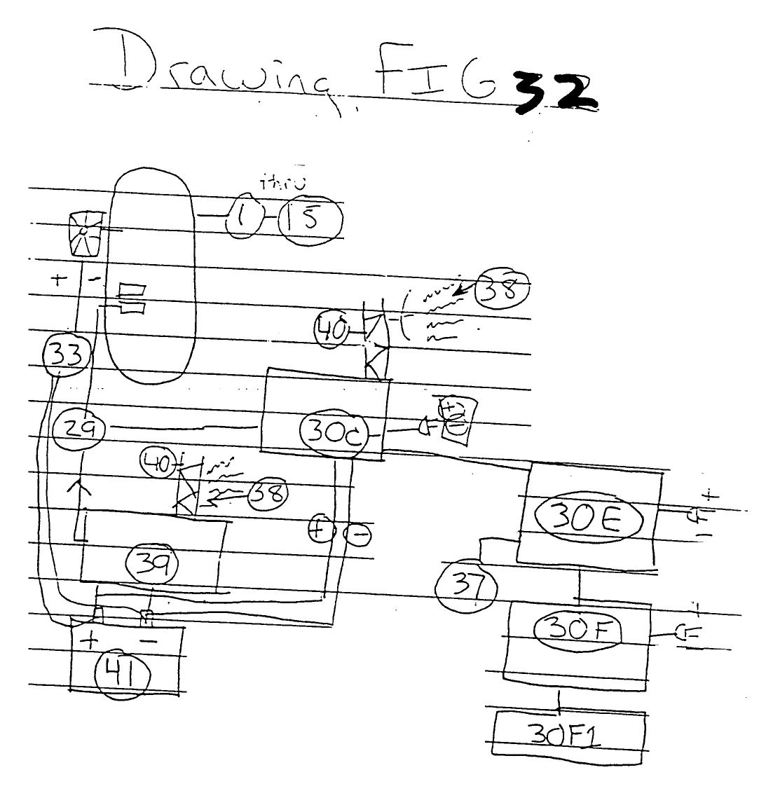

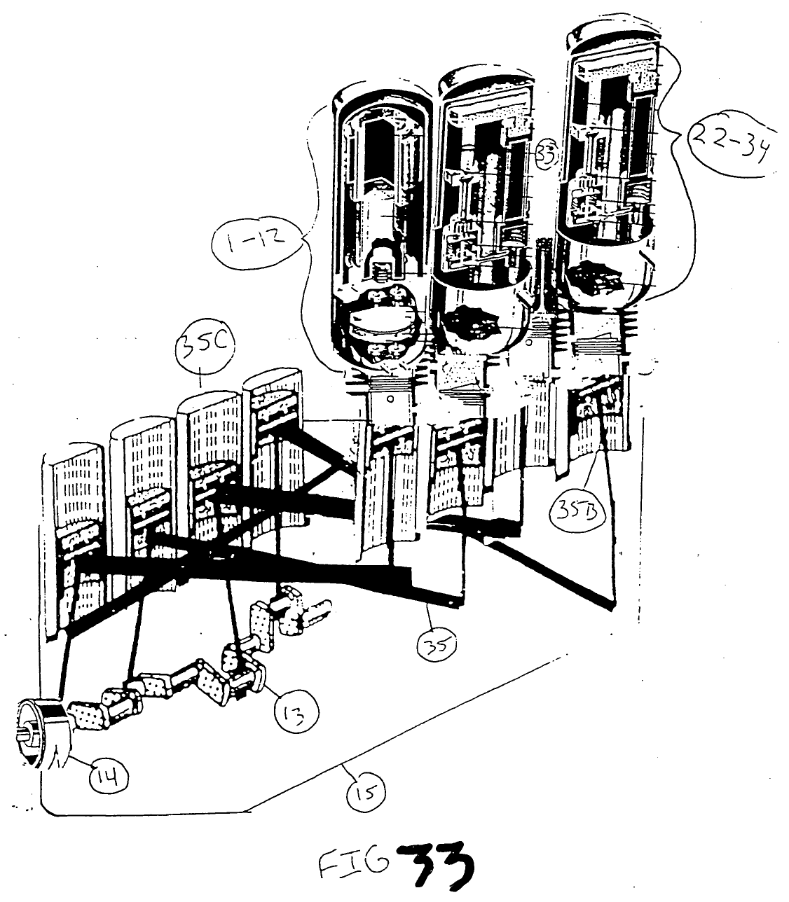

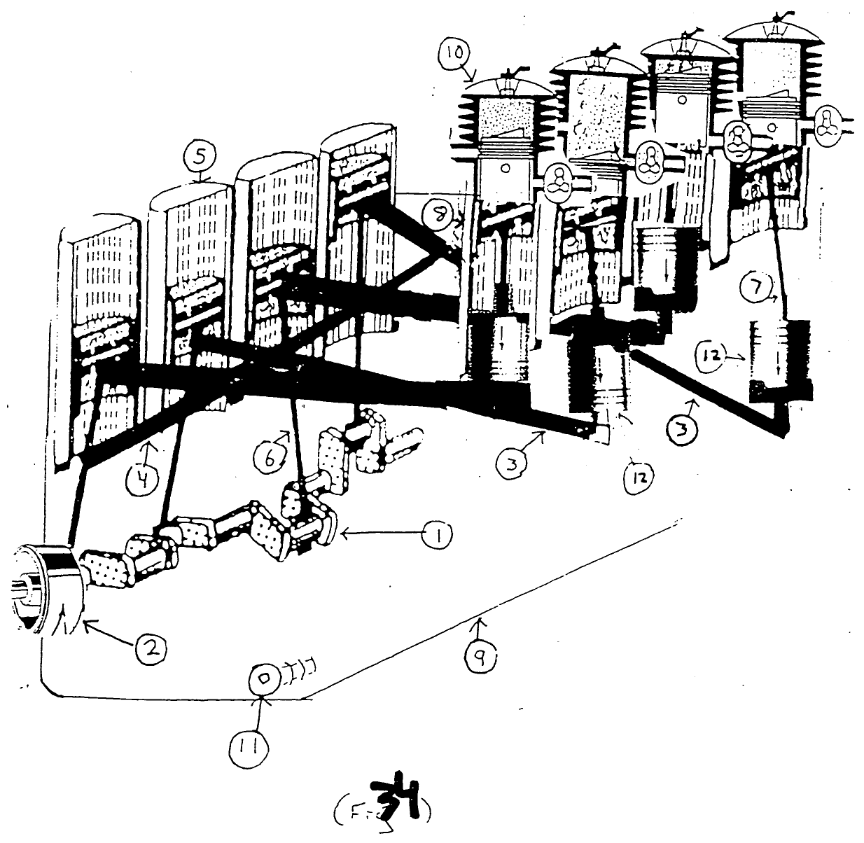

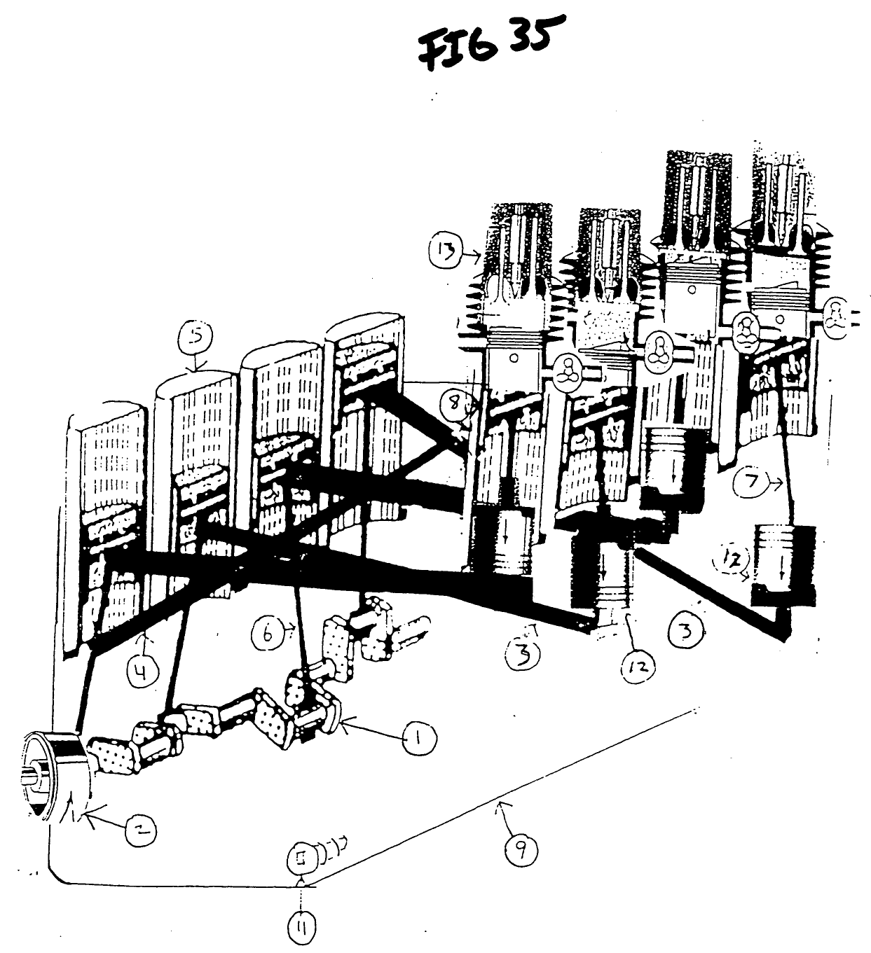

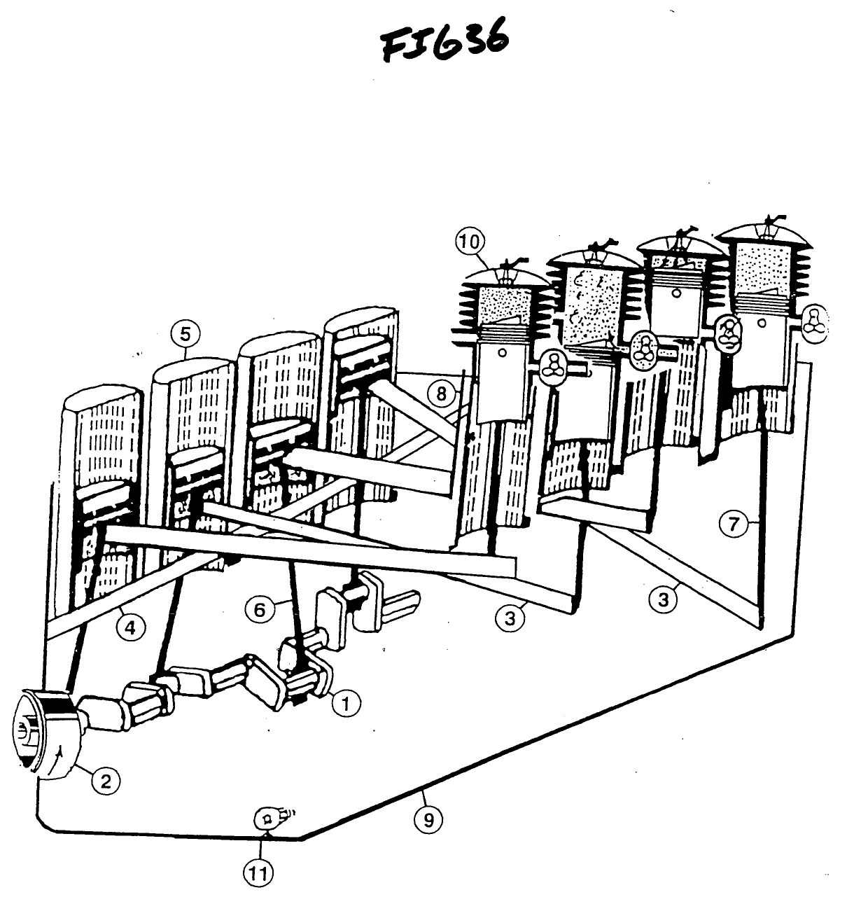

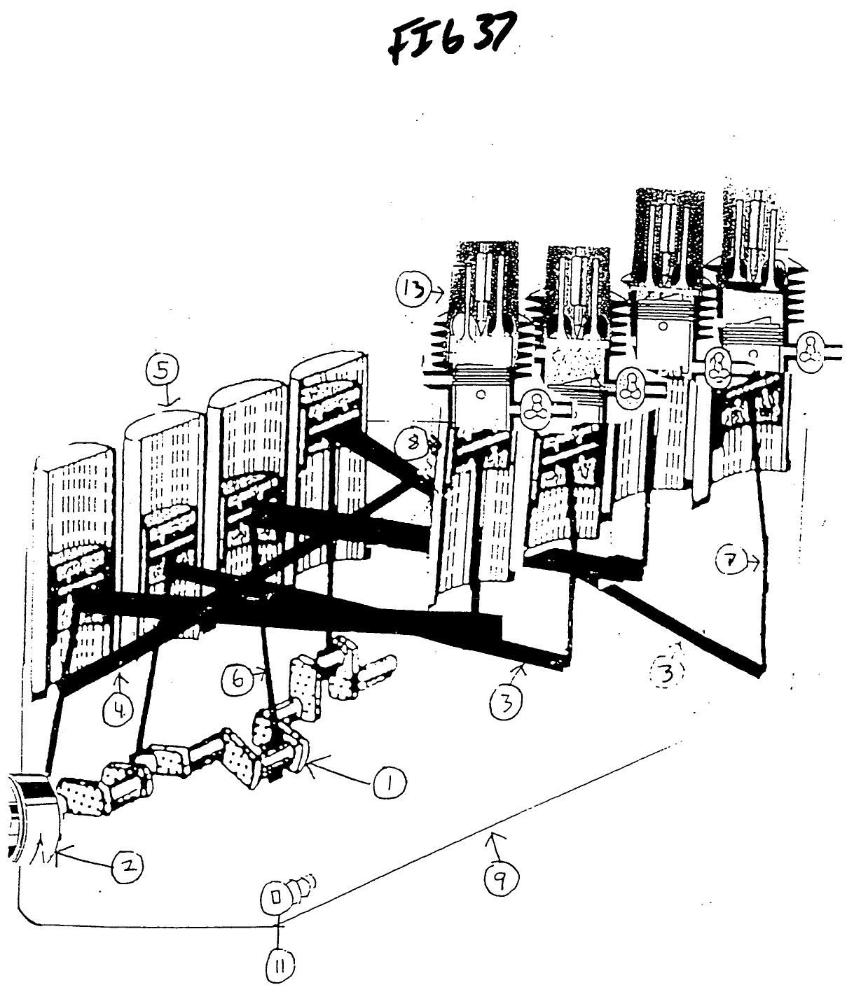

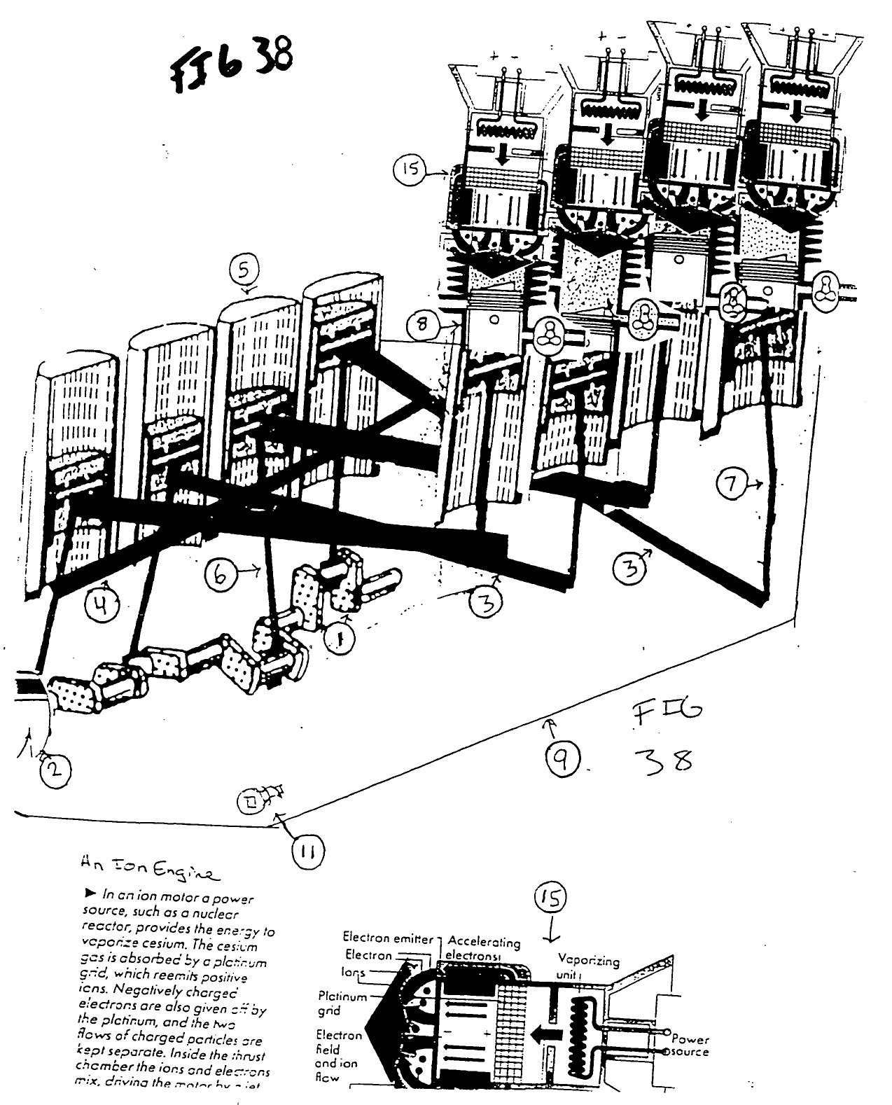

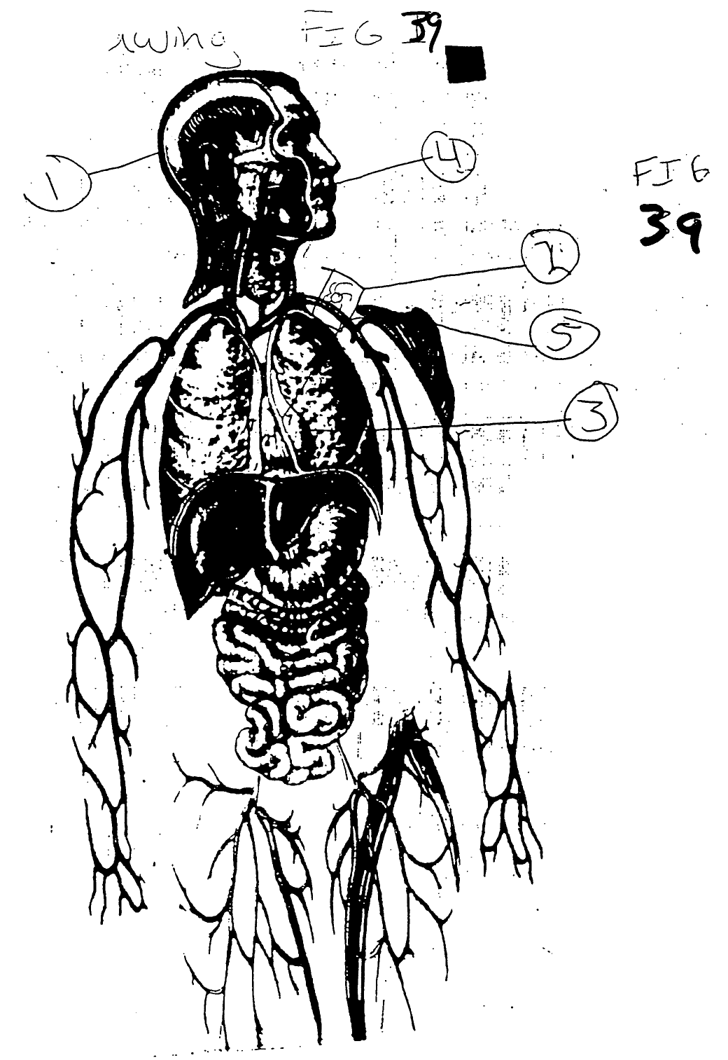

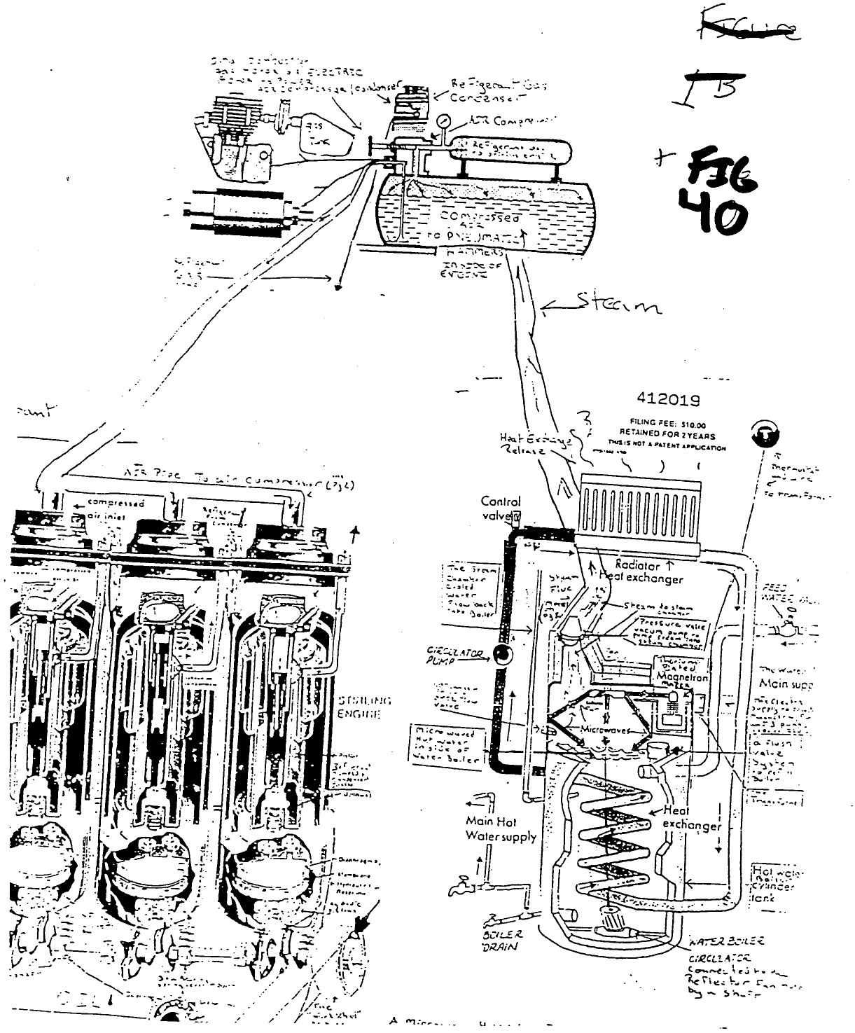

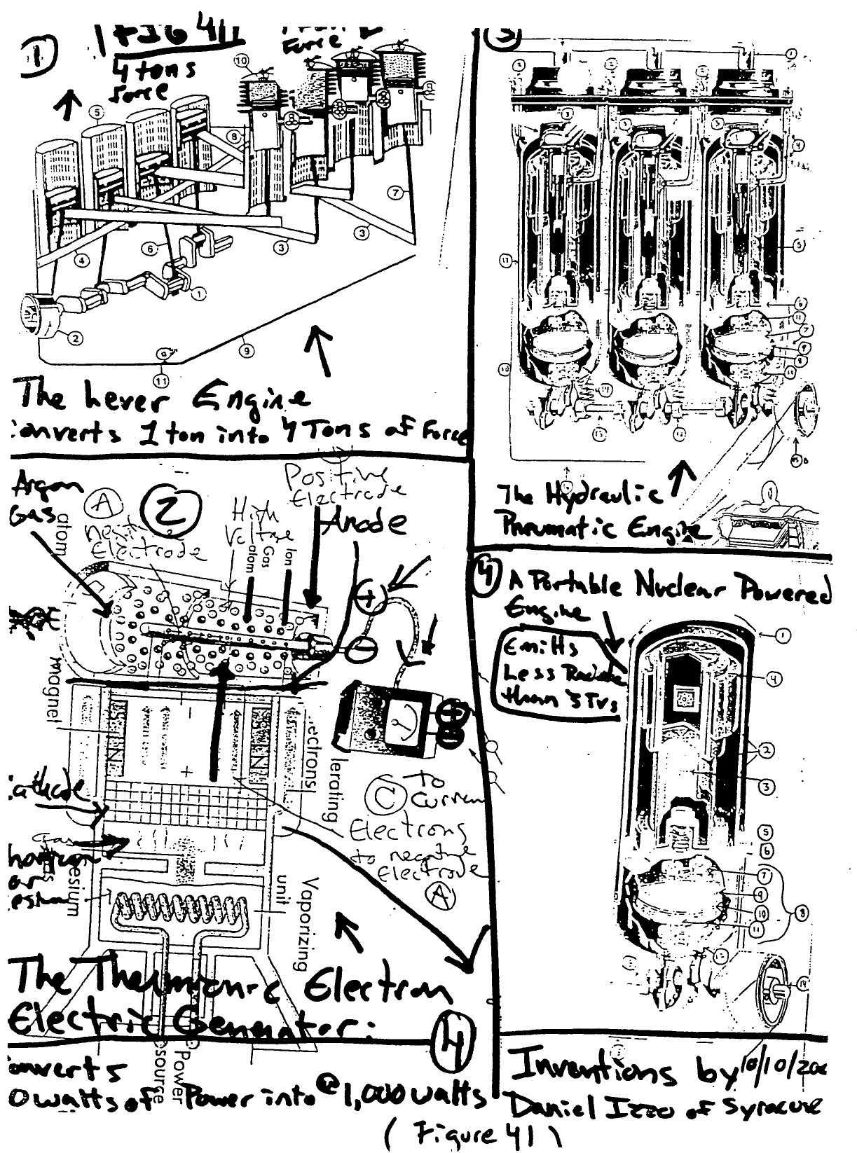

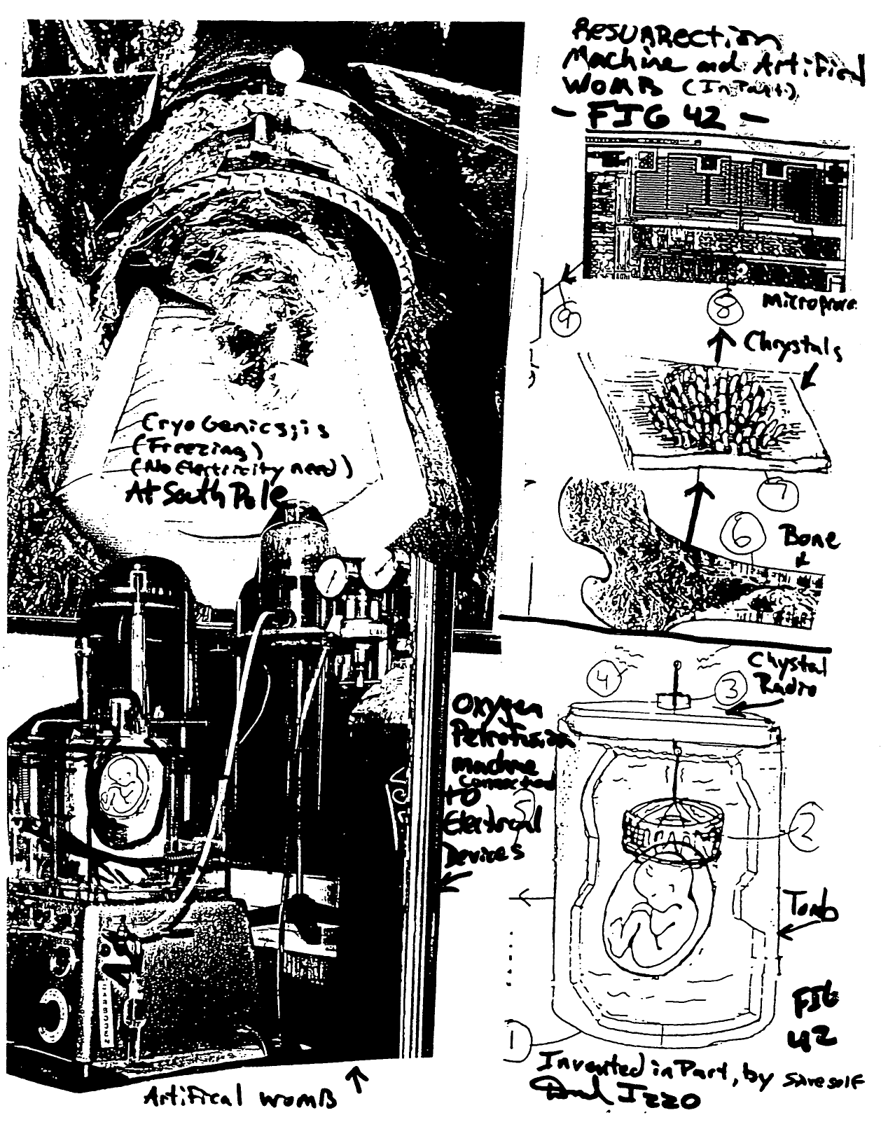

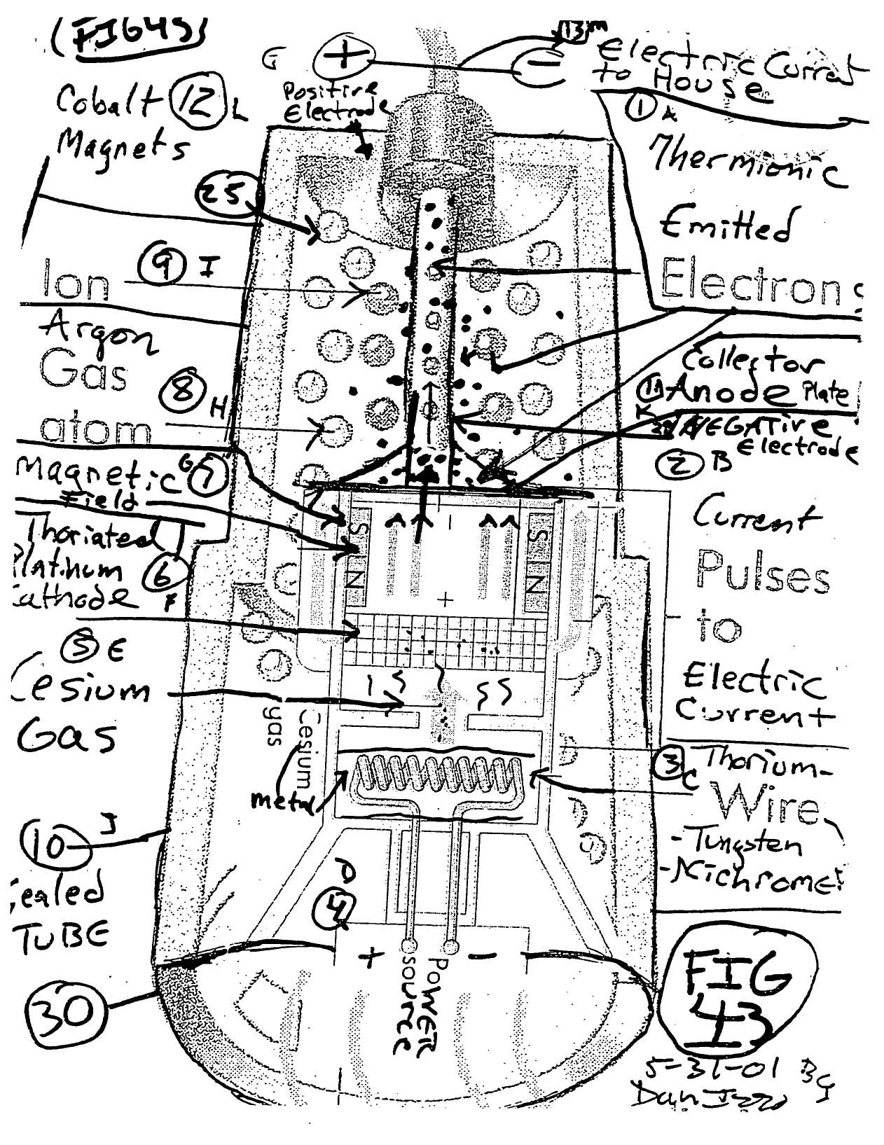

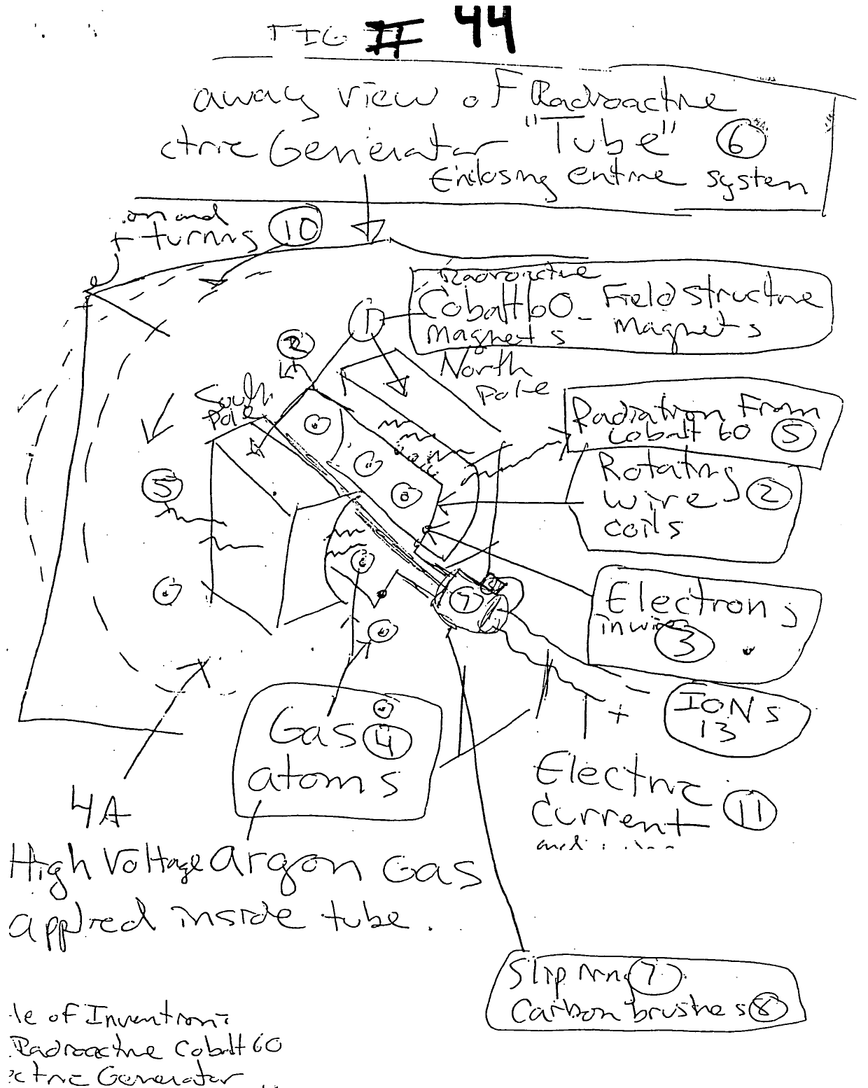

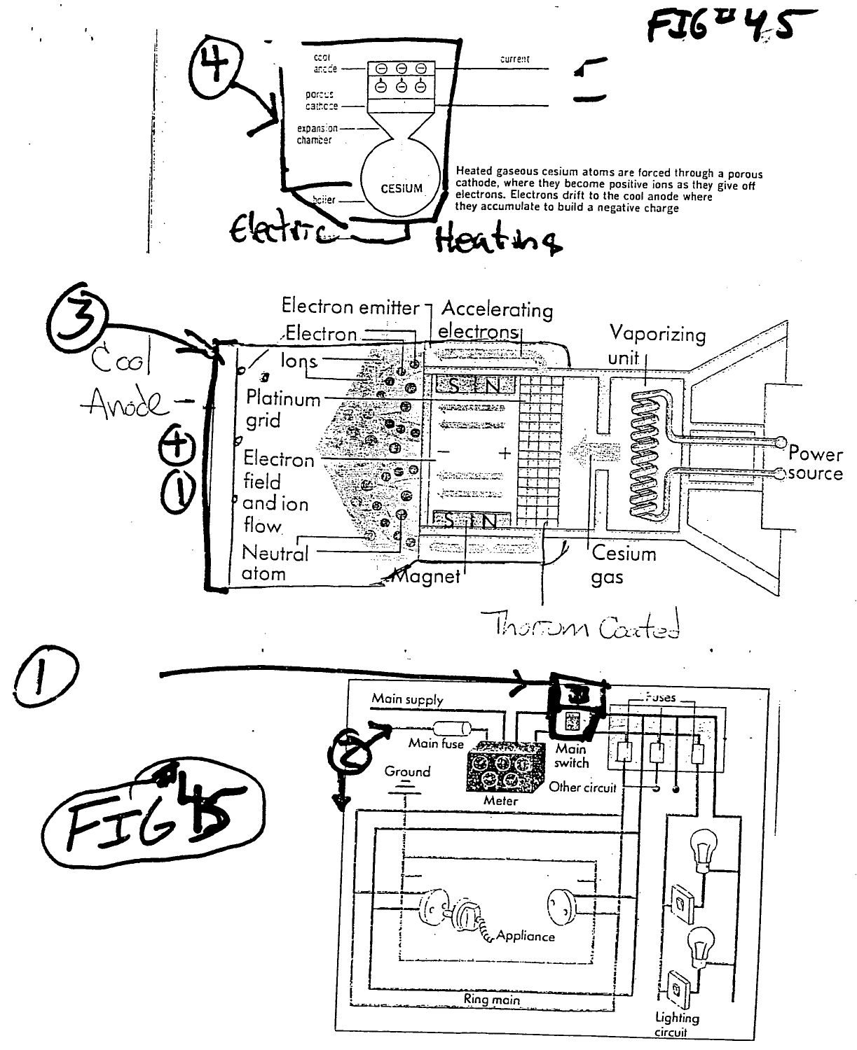

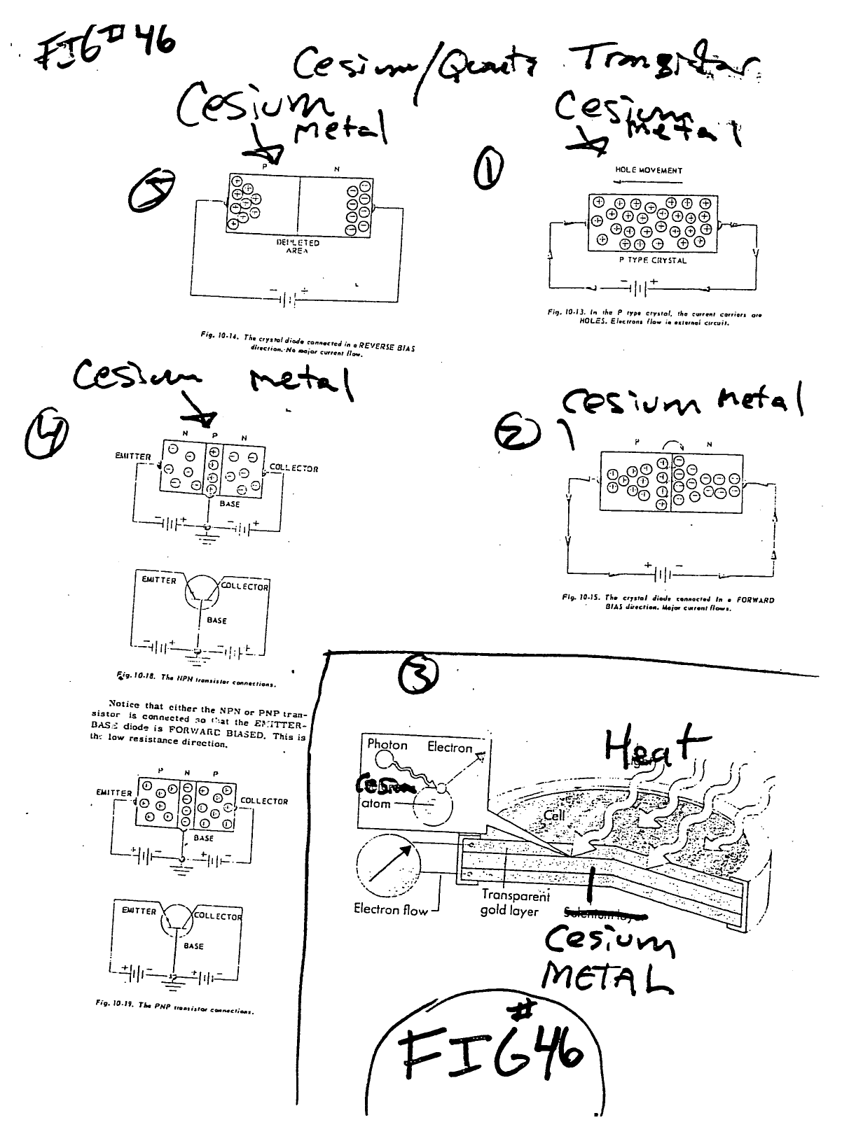

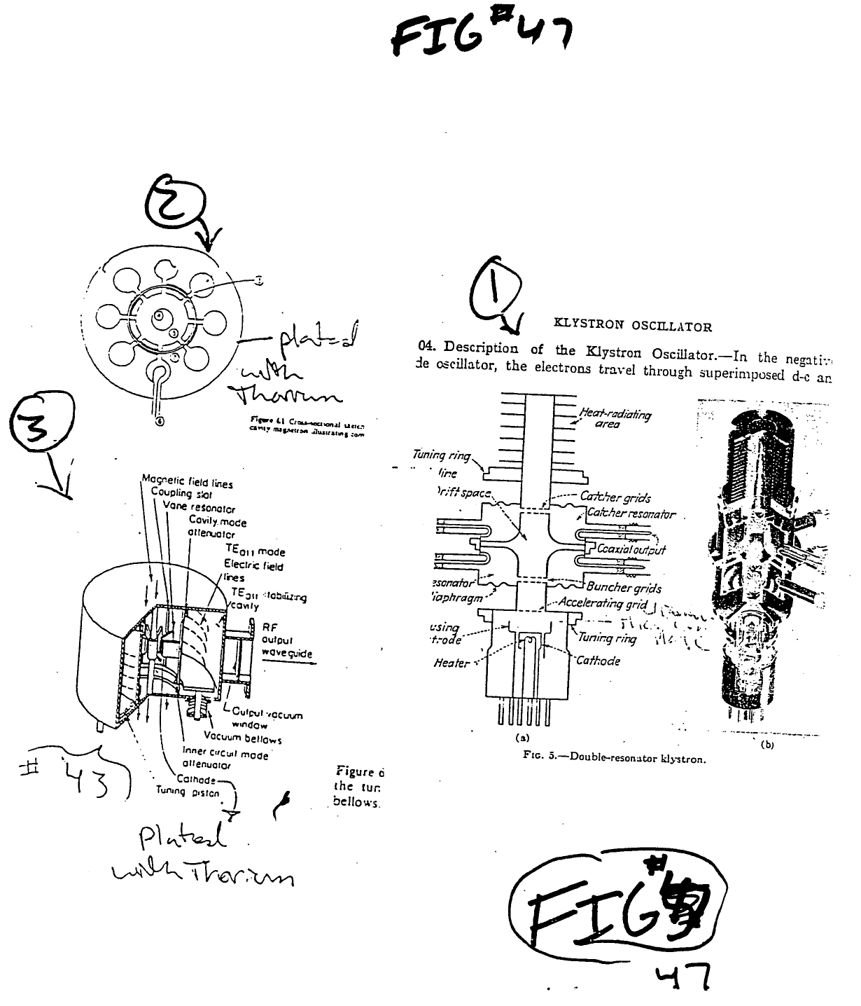

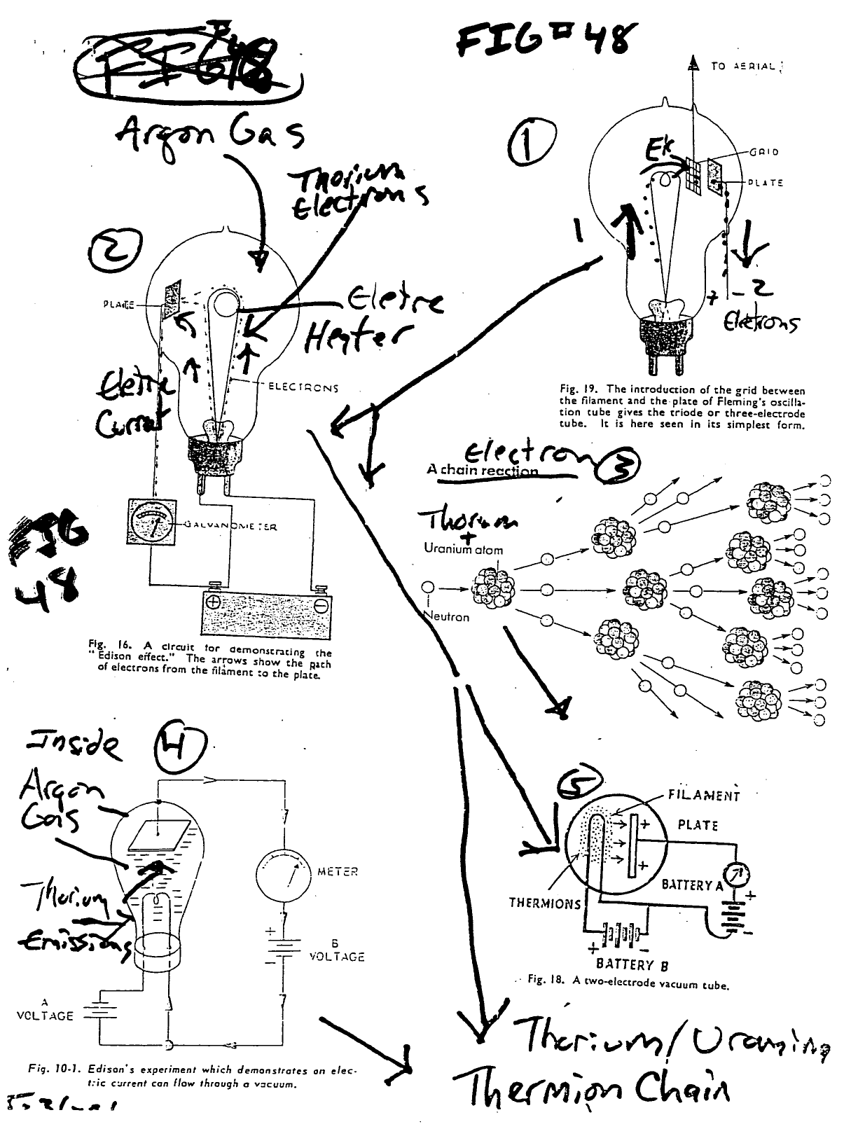

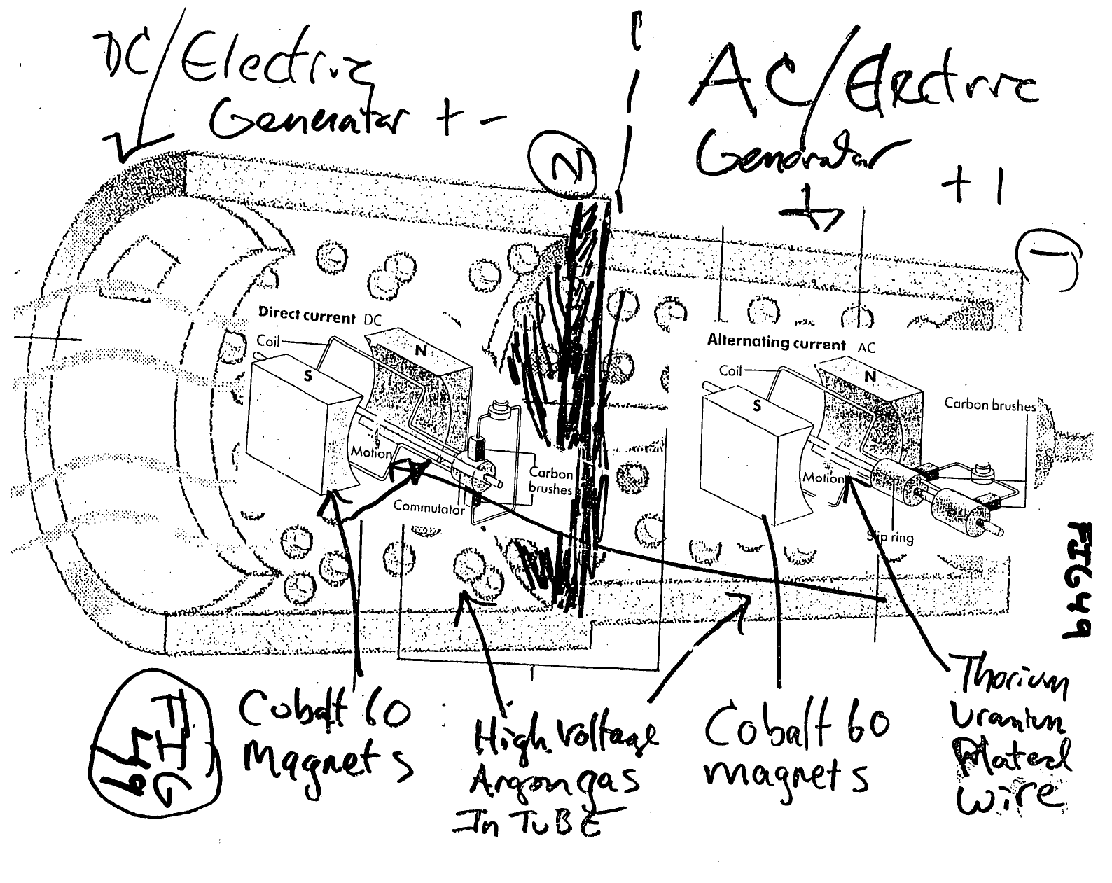

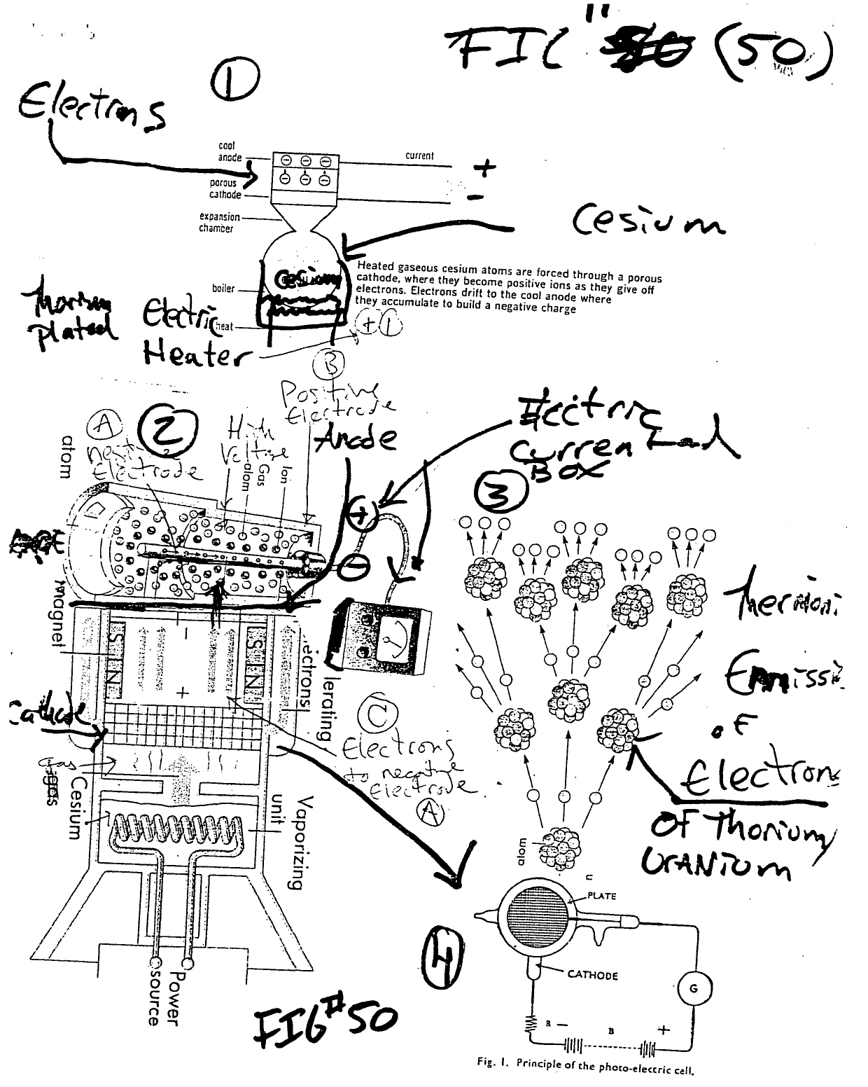

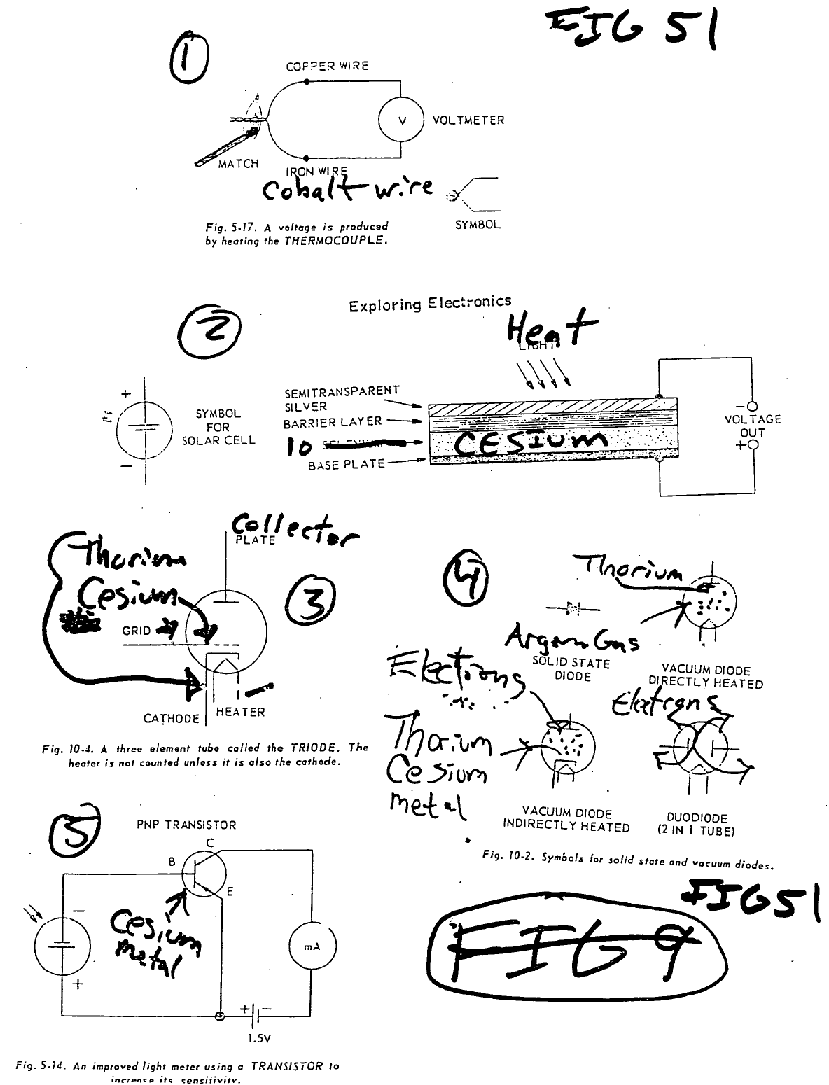

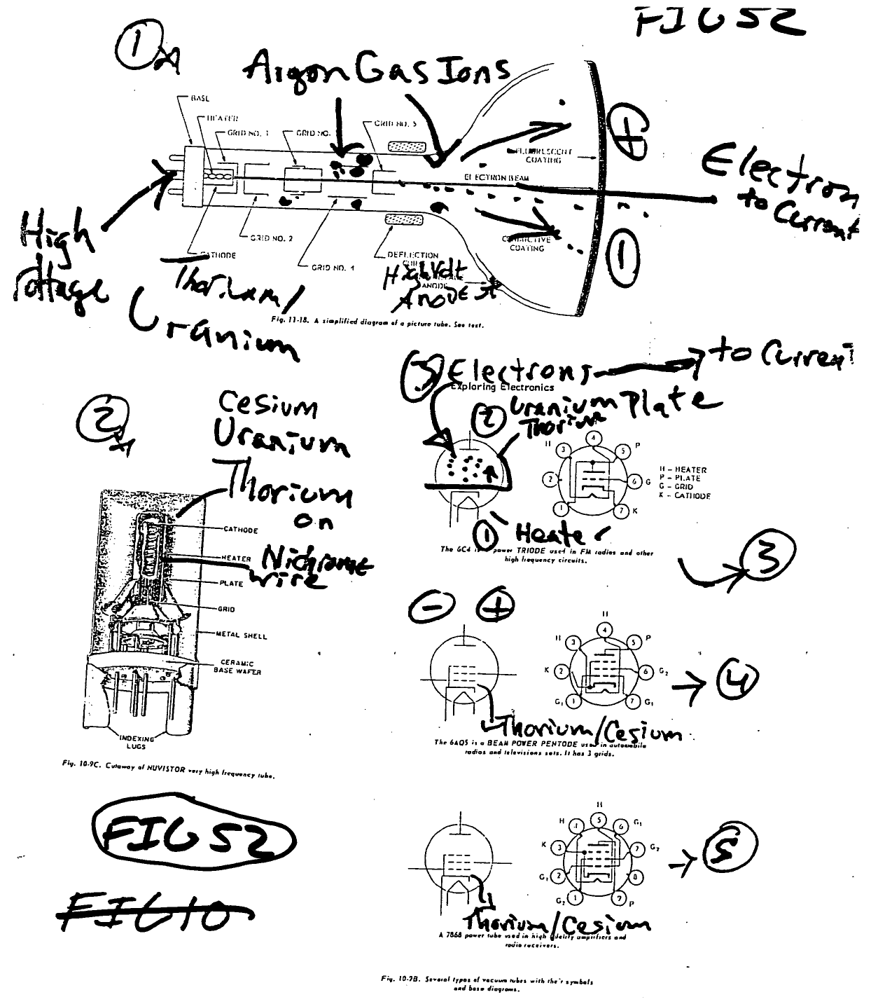

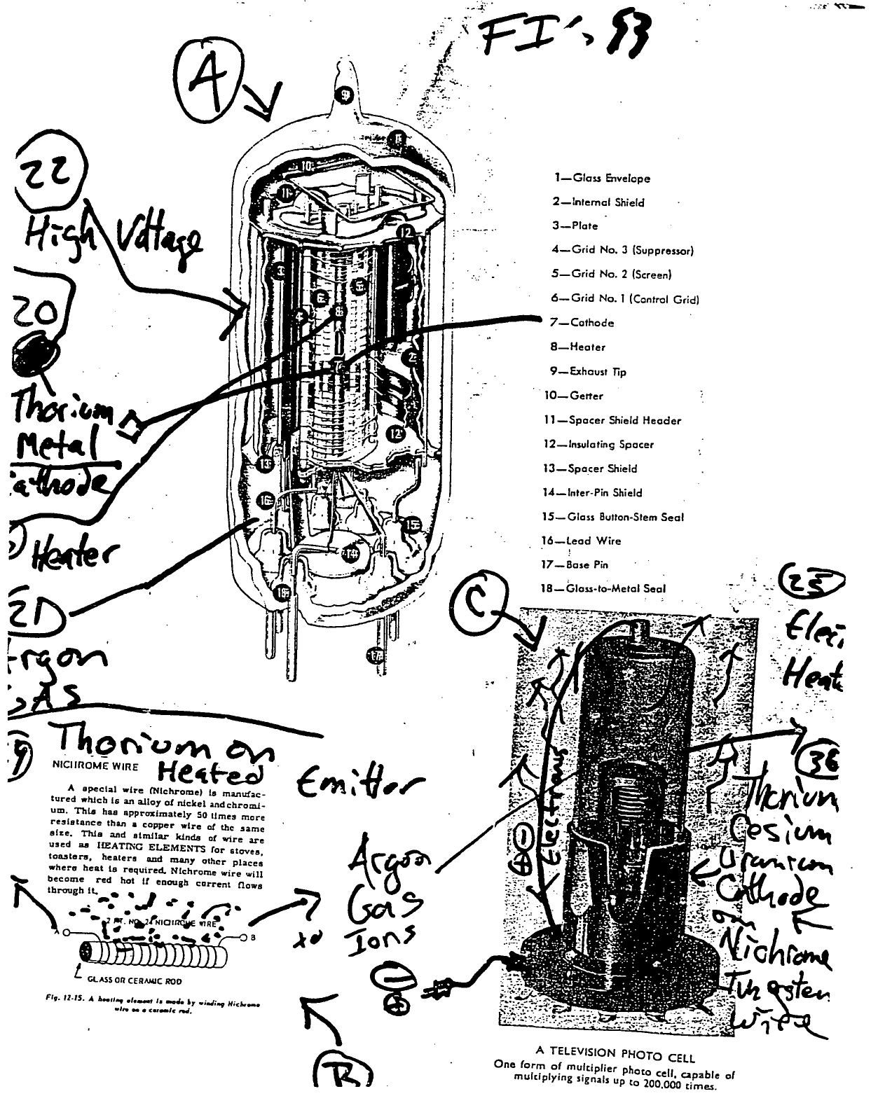

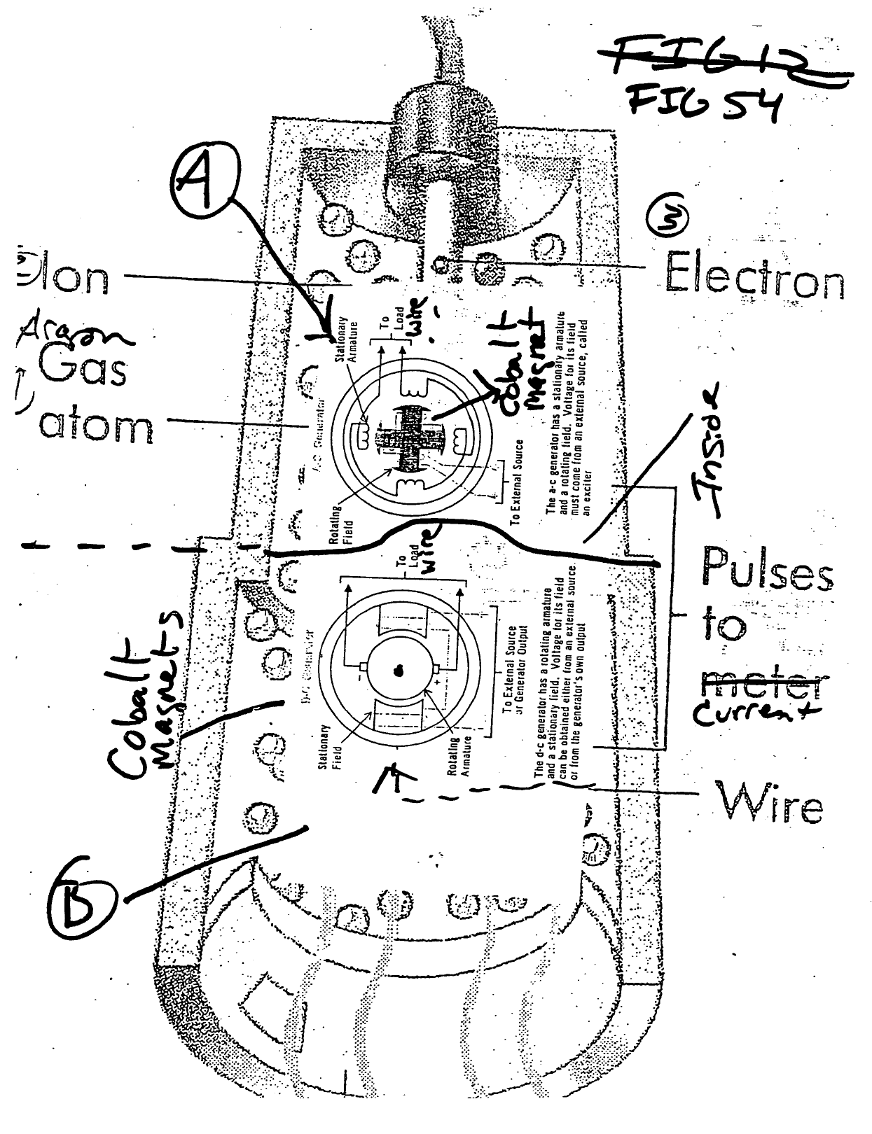

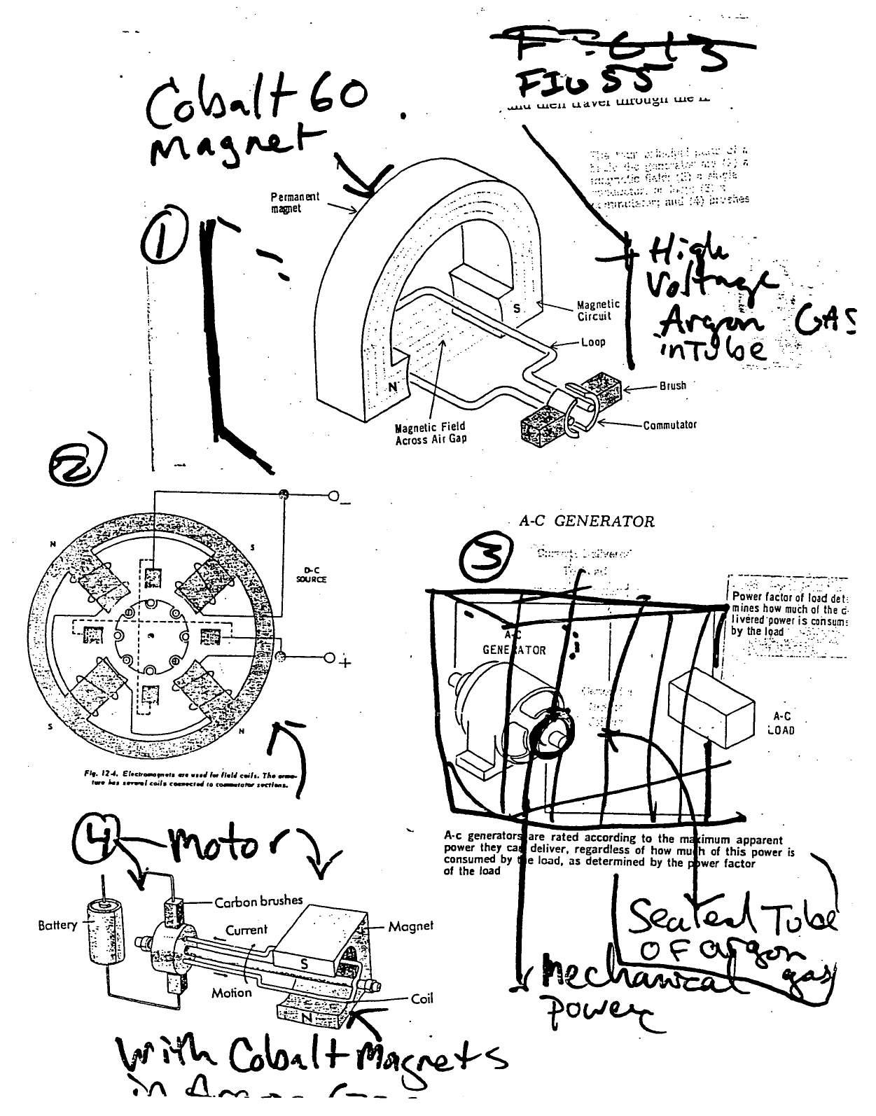

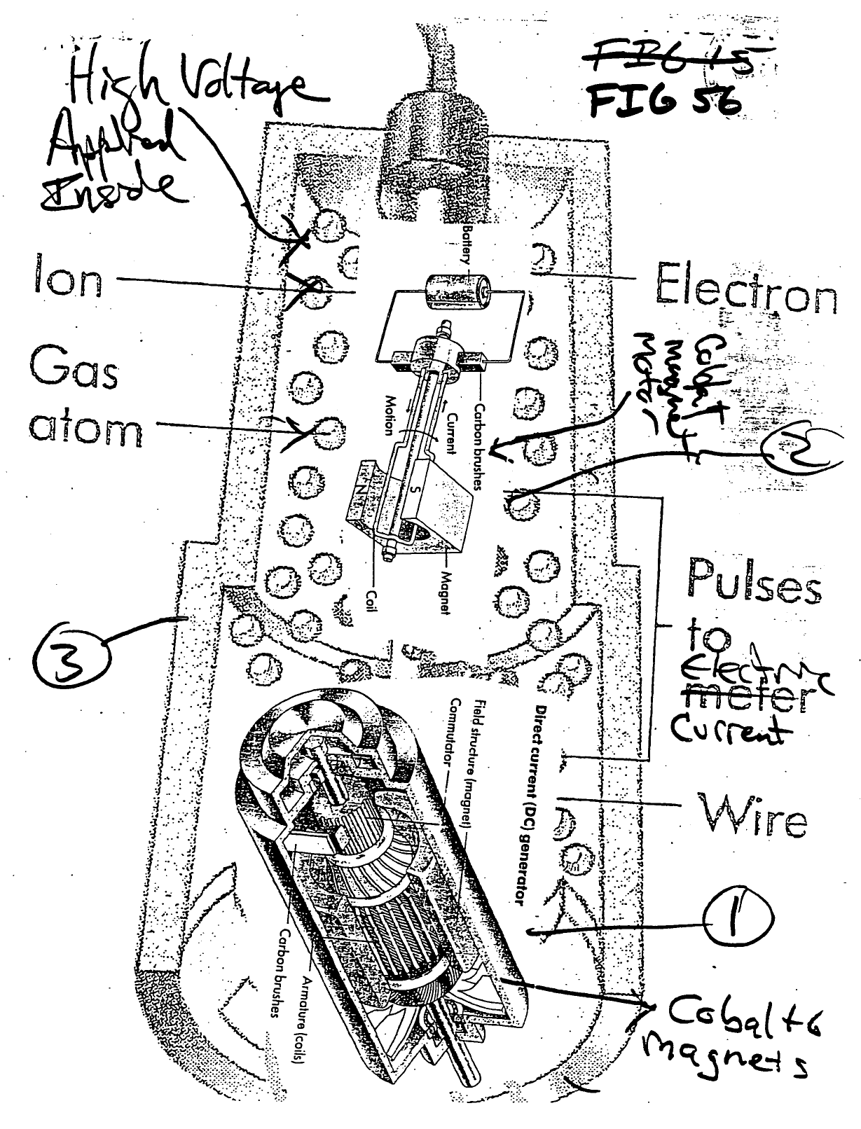

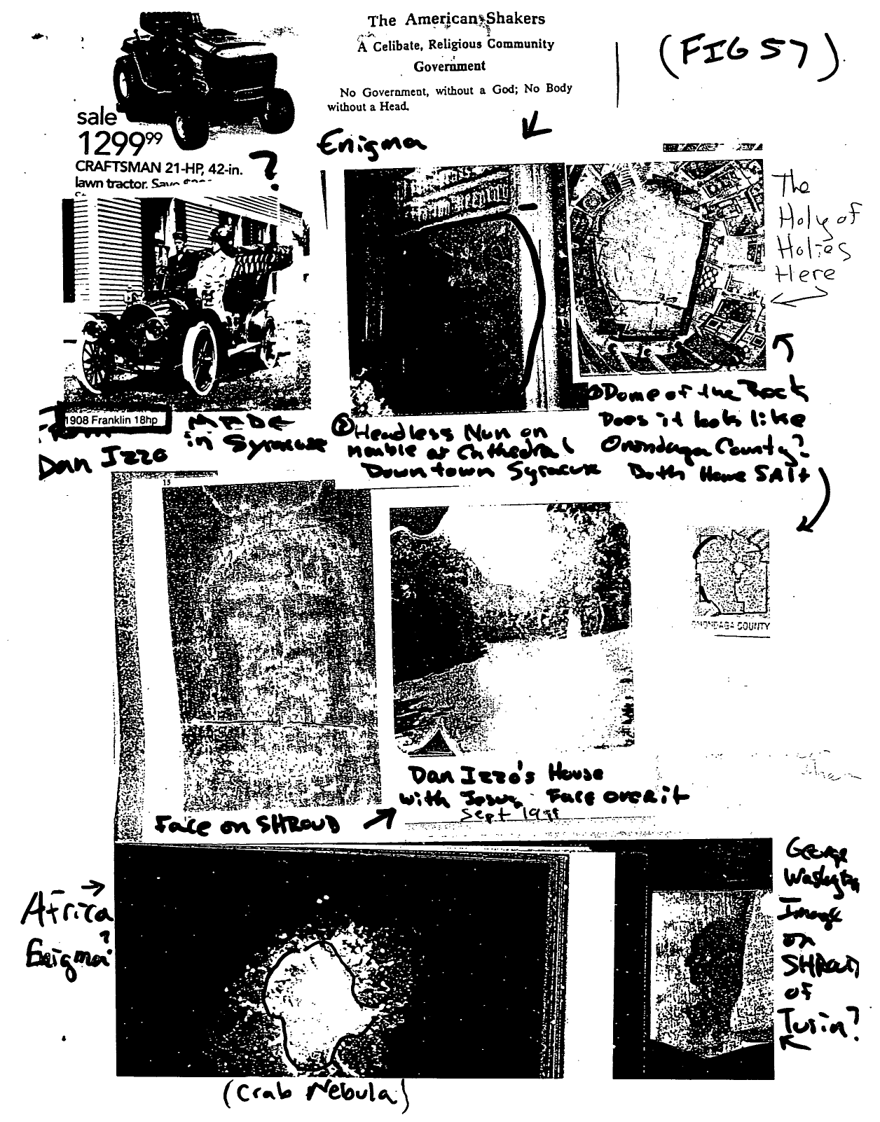

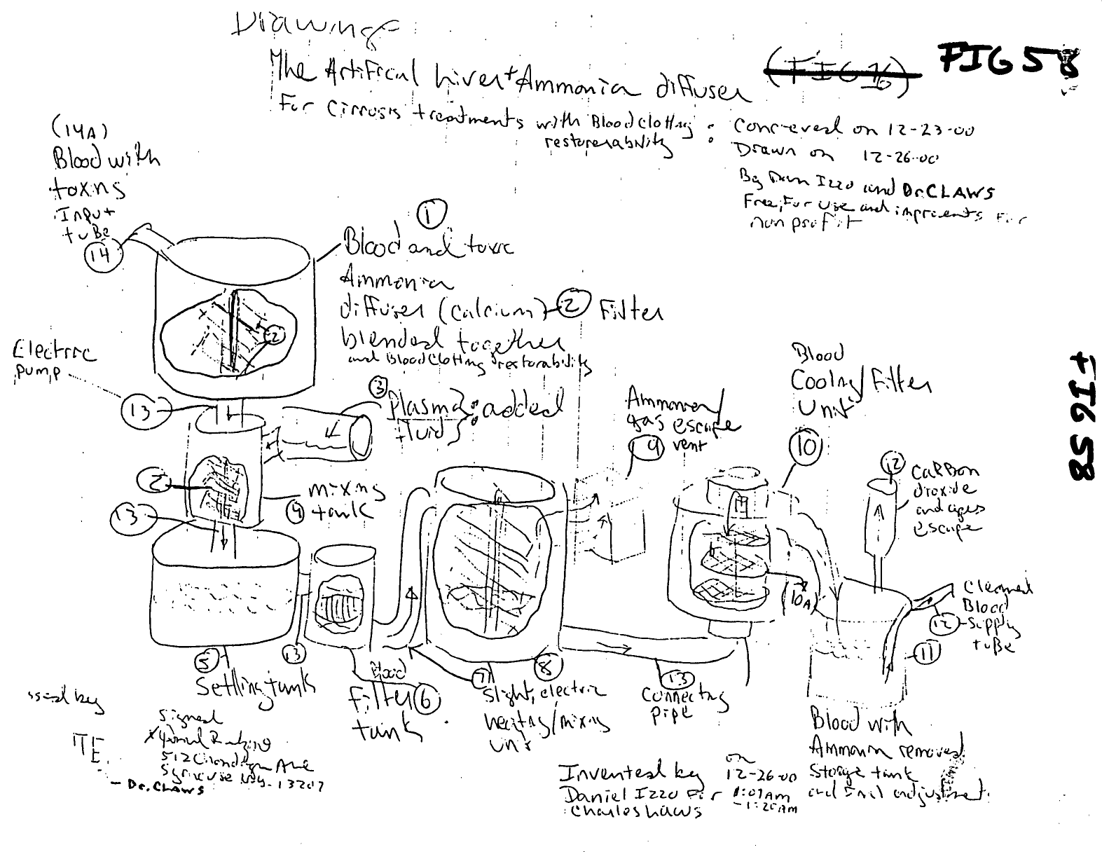

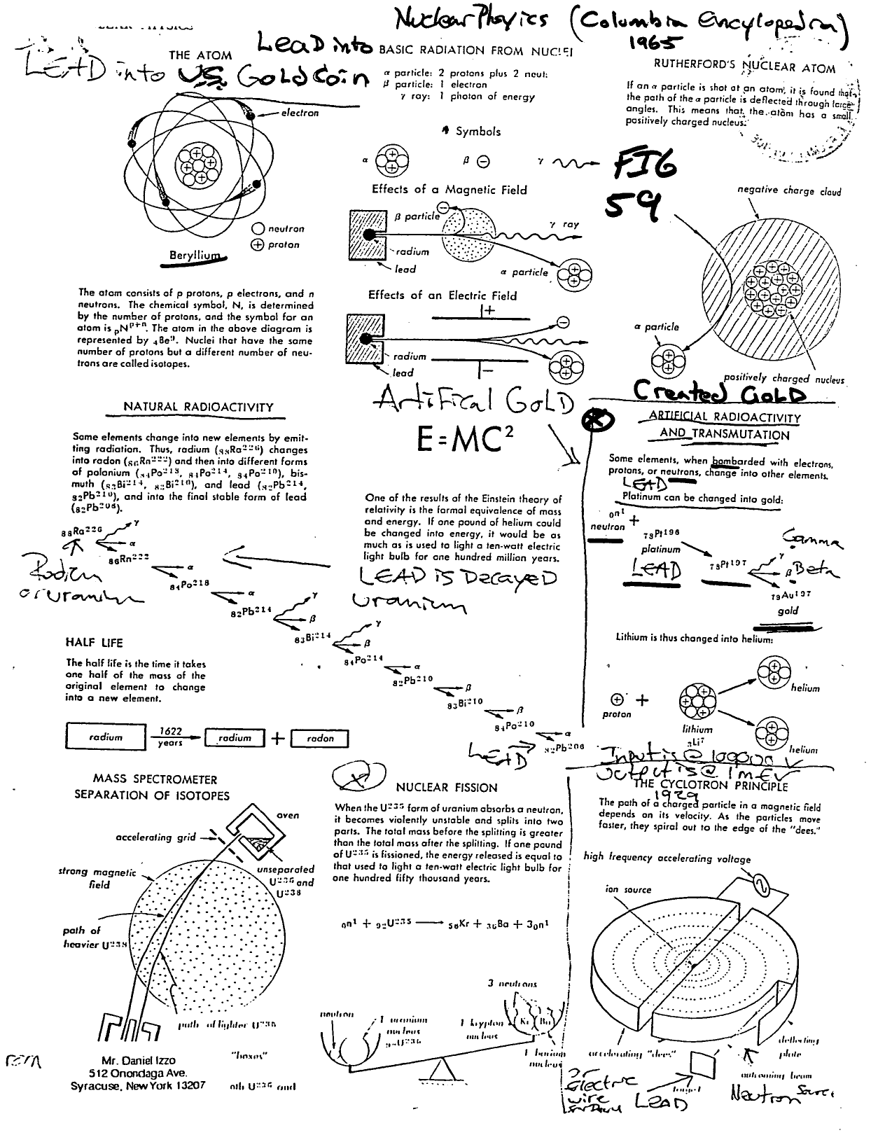

1) Is a drawing of the Resurrection Tomb holding the container of preservative; holding the dead person's body and connected to electrical and energy apparatus contained in the Resurrection Tomb consisting of concrete/cement molded manhole building/tomb with power source for the dead/dying human beings.

2) Sealed entrance to building/tomb.

3) Double walled, vacuum sealed container with preservative medium that prevents decay of human tissue and a means for tissue nutrition and oxygen.

4) Depicts a power source, of a portable nuclear powered engine and electric generator and batteries for storage of electric power.

5) Depicts a radio and communication system with antenna connected to power source.

6) Turbine system that converts water and or wind power into electric/energy for the burial tomb.

7) Depicts intelligent robot maintainance worker.

8) Depicts a heating source.

9) Depicts an engine/pumping system

10) Depicts a means and apparatus that provides oxygen/nutrition t the surviving/growing human tissue.

11) Depicts electrical apparatuses for monitoring communication and entertainment of the burial/chambers.

FIG. 2

1) Depicts a dead human being inside of a container containing tissue preservative fluid; that has a radio wave crown.

2) Connected to the head of the person; with an antenna that connects to a radio.

3) That is either electric powered or a crystal radio; that can collect and store radio wave energy that all 1-4 fits inside of a piece molded man hole/building burial tomb.

4) Radio waves

5) Building/tomb.

6) A human being's bone containing silicon, phosphorus and boron that is ground up.

7) Grounded up elements and are used to produce a computer electric circuit that is placed inside of a computer.

8) (Harry Quartz and Silicon crystals that oscillate at the frequency of the dead person's life/energy force.)

9) With a human skeleton frame

10) With a few bones missing waiting to have artificial muscles and intelligence restored to it's material. To become conscious again with it's old stored thoughts, mannerism and memories.

FIG. 3

Depicts two human beings (in fetal form) being kept alive in a jar of fluid.

1) Awaiting the day that new nerves regenerate in their bodies to regain consciousness once again. Please note: When we die, ” not all of our body is “dead” mainly our nerves and brain has “burned out” and “died”, when we die. The rest of our body is OK for several days.

FIG. 4

Depicts a human skeleton that will be filtered with new artificial nerves; muscle and conscious together with organic compounds and nutrition.

FIG. 5

Depicts a human skeleton being filled with artificial nerves; muscle and eyes, preparing for a conscious resurrection.

FIG. 6

Depicts the preserved brain and nerves 1 that was from FIG (1). The preserved brain and nerves have been treated with nerve growth hormone that was cultured from olfactory nerves and other nerve hormones.

FIG. 7

Depicts arteries and veins far carries blood 1 or energy far artificial muscles.

FIG. 8

Depicts a human skeletal being fitted with artificial muscle that expands and contracts with data inputs. The artificial muscle used is silicon with elements and minerals used to expand and contract silicon and artificially grown organic muscles.

FIGS. 9 & 10

Depicts the Portable Nuclear Powered Engine and Electric Generator that produces energy for the unconscious human being's home and the dead human being's tomb, see CIP for specifications.

FIGS. 11 & 12

Depicts the Portable Nuclear Powered Engine and Electric Generator obtaining cosmic radio wave energy and is connected to the human material computer (30E; 30F;37) See the CIP specification for Ser. No. 09/205,415 and CIP application Ser. No. 09/177,760.

FIG. 13

Depicts the Cement Molded Building and Tomb (19B) being powered by the Portable Nuclear Powered Engine and Electric Generator, see CIP Application Ser. No. 09/177,760, and CIP specification for Ser. No. 09/205415.

FIGS. 14 & 15

Depicts the Microwave Hot water (40) Powered Heating System with Thorium plated Magnetron maser that provides heat for the building and tomb. See U.S. Pat. No. 6,064,047.

Ser. No. 10/181,974

FIG. 19) Depicts the invention's Citizen Band Free Public Radiowave Telephone System's unit A and B

1) Audio input, microphone.

2) Frequncy programmer number pad to frequency crystal oscillator.

3) Programmed radio frequency to signal number display showing 4.725088 mhz, 47.25088 mhz and 472.5088 khz, usable in the U.S. when the goverment (FCC)allows common carrier use of radio frequency numbers similar to a person's telephone number, from 200 khz thru 999 khz, 0.000010 mhz thru 9.999999 mhz, 0.0000010 mhz thru 99.999999 mhz. (with public radio stations frequencies reserved).

4) Transmit and recieve antennas.

5) Keyboard to computer input.

6) Speaker.

7) Video Monitor/TV monitor.

8) Fax machine/printer.

9) Data input and computer storage data unit.

10) Electric plug input/output.

11) Telephone line for wire service.

12) Cable input/output wire.

13) Video/Audio camera that is removable.

14) Source of communication antenna for (a) and (b) Radiowave reciever and transmitor

15) Propogated Radiowave from devices A and B.

16) Radio reciever speaker.

FIG. 19B) Depict's the invention's;

(1) audio input with cable wires

(2) input amplifier;

(3) radio frequency amplifier;

(4) modulated amplifier or a frequency modulator;

(5) modulated amplifier or a frequency modulator-master oscillator

(6) Radio frequency amplifier;

(7) Radio Frequency amplifier;

(8) Radio Frequncy amplifier;

(9) Crystal oscillator that is programmable;

(10) Antenna;

(11) Pre-selected circuit;

(12) Frequency changer;

(13) Intermediate Frequency amplifier;

(14) Frequency detector,

(15) Low Frequency amplifier,

(17) Antenna

(18) local oscillater,

FIG. 20) Describes the invention's stream paddlewheel and electric generator, wherein the wind/river water flow (2) causes multipule paddlewheels (3) to turn a shaft (4) that turns and drives an electric generator (5) and the unit (1) produces electric current that is stored in the electric storage unit (6) being maintained/operated by the “Artificial Muscle Robot” of figure (2) part (10).

FIG. 21) Describes the invention's “Artifical Muscle Robots” artificial hand and the invention's artificial muscle hand having (1) one with artificial muscle (2) that is connected to a (bone) frame (8) with movable jionts (4) and the artificial hand (1) is covered and protected with a material (3) that permits movement. The invention's artificial muscle (2) will expand and contract with force, using electric current by way of spring wires (6) that is within the artificial muscle (2) that is weaved and patterened and layered like a real human hand having sensors (7) for input/output signals through a central jiont palm (5).

FIG. 22) Describes the invention's, “Artificial Muscle Robot” (10) that has a computer brain (9) with optical sensors (11) and an input/output communication device (12) and recieves transmits radio communication (17) and the device (10) has arms (13) with artificial hands (1). wherein the robot (10) is powered by one of the invention's, “Portable Nuclear Powered Engine (15) and (16) and other electric inputs (18) and can move and perform handwork (1) (7) (6) of many tasks Human hands can perform to make Human life easier.

FIG. 39) Describes the invention's “Artificial Heart Assistor Pump” and the “Zinc-Clove Leaf Oil Paste” 1) A Human Person 2) An Artificial Heart Assistor Pump that is electric or nuclear powered. 3) A Human Heart. 4) The Zinc-Clove leaf oil paste on a

FIGS. (23) (24) (25) Describe the invention's, “Portable Nuclear Powered and Electric Generator” that comprises of a Stirling type engine having a doubled walled capsule cylinder (1) that is filled with helium gas (2) and contains a floating piston (3). At the top of the floating piston (3) is a pellet of radioactive material (4) that heats the helium (2) to about 1,200 degrees F., This very hot helium (2) expands rapidly, then forcefully drives down the piston (3) to the bottom of the cylinder (1) where the tempature is almost 1,100 degrees F., lower. As the piston (3) moves down it forces some of the gas out through a value (5) in the cylinder floor near the spring bellow (6). The floating piston (3) acts and pushes against the upper hydraulic jack (8) piston (7). The upper hydraulic jack (8) piston (7) will push against the helium resvoir (9) and will compress the helium gas and pushes against the helium resvoir (9) and will compress the hydraulic fluid resevoir (11) that will force the hydraulic jack pumps (8) lower hydraulic pistons(12) that will force and act upon a larger load than was originated upon it, and will push this force against the crankcase (13) that will turn and turn the load bearing work wheel (14). The invention's oil pan (15) is filed with oil. The work wheel (14) will turn an electric generator (16). The entire device is contained inside of a magnetic bottle (17) that has a coil of a magnetic field metal (18). The magnetic bottle (17) is contained in a ceramic box (19) that is covered with lead plates (20). A battery (21) is used to hold the electric current in storage for use of the electric systems of the invention.

FIGS. (27) (28) (29) Descrbe the invention's “Portable Nuclear Powered Engine and Electric Generator” consisting of a Tidal regenerator engine that has a double walled capsule cylinder (1) containg a signal computer (30) that signals an electromagnet (29) that is activated to attract another magnet (29b). The movement expands a bellows (22a) that forces a little water (26) up a tube (31) into a water boiler (24) where heat from a radioactive source (23) and or a thorium plated magnetron maser (33) protected by a window of quartz (34) will vaporize water (26) into steam (32). The steam (32) is heated further inside of a super heater tube (21). The steam (32) then pushes another bellows (22b) that, in turn, sends a pulse of hydraulic fluid (28b) through the hydraulic jack piston pump (28) and will compress the hydraulic piston (28c) that will force and act upon a larger load than was originally acted upon it, and also force a lever (35) and together will push this force agaist the crankshaft (13) that will turn and turn the load bearing work wheel (14). The engine's oil pan (15) is filled with oil. The work wheel (14) will turn an electric generator (16). The entire engine is contained in a ceramic box (19) that is covered with lead plates (20) and barium plates (20b) and the ceramic box (19) is located within a cement molded building (19b) and shall contain barium radiation absorbers and power extinguishers (20c) that shall discharge and absorb radiation when the extinguishers (20c) sense radiation leakage.

FIG. 30) Describes the invention's “Portable Nuclear Powered Engine and Electric Generator”, cylinder housing (1) operating inside of a magnetic bottle (17) that is contained in a ceramic box (19) that is covered with lead plates (20) and barium plated (20b) and is located within a cement premolded building (19b) that contains the barium radiation absorbers and powder extinguishers (20c).

FIG. 31) Describes the invention's “Portable Nuclear Powered Engine and Electric Generator” cylindrically shaped housing (1) having water (26) and a source of electric power to be amplified and converted into microwave energy (36) and a source of generating microwave energy (33) having its magnetron cathode (33a) tubes plated with radioactive nuclear materials as a source of amplifying the cathode rays that will be coverted into microwave energy to provide an output (33b). A computer (30) that uses either cable wire (30b) and or transmits data by radio waves and television KHZ-MHZ-GHZ waves (30b) to a radio and television reciever (30c) that is connected to a computer (30e) that is connected to a magnetic video/audio tape recorder (30f) for recording computer data inexpensively, wherein 1 vhs magnetic tabe (30f) can hold 1.5 gigabytes of computer data.

FIG. 32 Describes the invention's “Portable Nuclear Powered Engine and Electric Generator”, cylindrically shaped housing (1) and thorium plated magnetron that emits microwave energy (33) and a magnet (29) recieving zzzzz930c) (39) cosmic radiowaves (38) and converting the energy into usable electric current (40) by using a crystal radioset (39) with an antenna (40) sensitive at collecting radiowave energy (38) and using and storing it (41) inside of a battery. The cosmic radiowaves (38) shall also be recieved (30c) and recorded on magnetic tape (30f) and stored for analysis of fluctuations and data pulses inside of the unit's (1-15) audio-video recorder (30f) and accessed by the computer key code (37)

FIG. 33) Describes the invention's Portable Nuclear Powered Engine and Electric Generator usage of lever bars (35) that will convert 1 ton of torque force into 4 tons of force, but a less distance.

FIG. 23) Details the ivention's “Portable Nuclear Powered Engine and Electric Generator” that emitts less radiation than 3 television sets.

Human Person's mouth that destroys microbes, such as Herpes virus.

FIG. 40) Describes the invention's “Hydraulic Pneumatic Engine” (1) and the “Microwave Hot Water Boiler Heating System” with a thourium plated magnetron (2) energy systems, using steam (3) from the microwave hot water boiler (2) to drive and help turn the hydrualic pneumatic engine (1) and electric generator (4) wherein the Artificial Muscle Robot of FIG. (10) assists in the operation of the invention's hydraulic pneumatic engine and microwave hot water boiler heating (5) energy system.

FIG. 41) Describes the invention's energy devices; 1) depicts the “Lever Engine” that can covert 1 ton of torque into 4 tons without gears/transmission. 2) depicts the “Thermionic Electron Electric Generator” having a power source, cathode, cesium, thorium, container with magnetic force inside, and a high voltage anode chamber with argon gas inside for output, wherein the input of 10 watts of electric power is amplified to about 10 watts of electric power output, for energy creation. 3)depicts the invention's “Hydraulic Pneumatic Engine” used for work functions. 4) depicts the invention's, “Portable Nuclear Powered Engine” that emitts less radiation than 3 tv sets and is used for work functions and electric power generation

FIG. 42) Describes the invention's rebulding and preseving Human persons who are deceased or suspended or are being revived into normal function, 10) depicts a means of cryogenic presevation, in the U. S. South Pole, Antartican territory 11) depicts a Human person in an “Artificial Womb” coverted from an oxygen petrofusion machine, wherein the Human's cells are preserved/protected and can grown into normal function. 9,8,7,6,) depicts a Human bone being crystalized and coverted into a computer microprocessor, for an “Artificial Muscle Robot” consciousness and for some normal function. 4,3,2,1) depicts a Human person inside a container that is filled with a prevervative medium that prevents decay and who is wearing a cyrstal radio crown to hear the living, awaiting the day of resurrection. (when we die, we can still hear, if there is ear drum function.).

FIG. 43) Describes the invention's “Thermionic Electron-Electric Generator” (30) that is used to generate the invention's electric power, that is contained inside a sealed tube (10)5 containg a source of input power (4)d that heats a thorium tungsten nichorme wire (3)c that is inside another sealed tube (20) that contains cesium gas (5)e that generate elctrons, wherein a thoriated platinum cathode (6)f obtains emitted electrons (1)a and emits more electrons inside a magnetic field (7)g having cobalt magnets (12)l; wherein an anode (11)k collects the elctrons and sends them to a argon gas ionizer chamber (25) having argon gas therein(8)h wherein high voltage electric is present (26) that ionizes the radiation of the argon gas (8)h and causes electrons to rush to one electron (27) and positively charged ions to the other electrode (27) and positively charged ions to the other electrode (28); wherein the electrons join other electrons emitted from the anode (11)k at the negative electrode (27); as the radiation and high voltage causes an electric current within the device (30) and sends it to the increased electric output to the invention's electric current supply (13)m

FIG. 44) Depicts the invention's electric generator having radioactive cobalt 60 magnets. and depicts radioactive cobalt60 magnets that cause electrons to move faster than iron magnets and produces more electric output per calorie than iron magnets and its output is increased by high voltage argon gas ionization inside the electric generator, wherein (1) radioactive cobalt fiel structure magnets create a magnetic field; wherein (2) coils of wire are mounted so that they can be spun around in a magnetic field with mechanical energy (10) input; wherein the entire device is inside a vacuum tube (6) that is filled with a gas (4) and charged to a very high voltage (4a) wherein the radioactive cobalt 60(1) ionizes the gas (4) spliting the elctrically neutral gas (4) into positively charged ions (13) and negatively charged electrons (3) wherein the positive ions (13) rush to one electrode (6a) and the electrons (3) rush to the other electrode wire (2) wherein this triggers an electric current from one electrode (2) to the other, (6a) while the mechanical energy input (10) turns the wire coil electrode (2) and creates electrical output (11) between the cobalt60 magnetic field magnets (1) wherein magnetic force and ionized gas of radioactive cobalt60 (1) magnets make electrons move in the wire coil (2) generating an electric current output, (11).

FIG. 45) Describes the invention's electric systems (1) connected to the invemtion's electric wiring (2) that provides amplified electricity; wherein the “Thermionic Electron Electric Generator” (3) (4) are a source of amplified electric power to the invention's wiring (2).

FIG. 46) Describes the invention's devices that convert heat energy into electric current (1) (2) (3) (4) (5).

FIG. 47) Describes the invention's Microwave Boiler, converting thorium/uranium electrons into microwave energy (1) (2) (3).

(1) depicts said magnetron microwave device, (2) (3) depicts said microwave magnetron and a klystron thats cathodes and andoes are plated with thorium and uranium and whose magnets are of radioactive cobalt., wherein the invention's microwave emitting device shall be improved by plating the magnetron's electric cathode with thorium and uranium that is radioactive, the anode is of magnatizable metal, radioactive cobalt, nickel or iron and the anode is plated with radioactive metal; wherein the operation of the invention's magmetron, a high D-C potent is applied between cathode and anode, setting up a radial electric field, wherein the combined forces cause electrons to take a spiral path and when the magnetron oscillates, the electric oscillations of the magnetron resonators set up an A-C electric fiel across the resonator gaps, wherein the thorium and uranium metal cathode, magnetic metal anode of cobalt and plated with radioactive metal in the magnetron sets up an increase in spiraling reactions when an electric current is applied and causes the magnetron output current to be many times the electric input of the invention's magnetron.

FIG. 48) Describes the invention's electric devices converting thorium, uranium, cesium tungsten, nichrome and argon gas atoms interacting and generating electron emissions into electric energy.

FIG. 49) Describes the invention's divices using mechanical energy input and obtaing a greater energy output of electric energy.

FIG. 50) Describes the invention's Thermionic Electron-Electric Generator, that coverts a smaller energy power input into a larger electric energy output.

FIG. 51) Describes the invention's devices converting heat energy and electro magnetic energy into a larger electric energy output.

FIG. 52) Describes the invention's devices converting a smaller electric energy input into a larger heating calorie output for room tomb air radiant heat.

FIG. 53) Describes the invention's devices converting thermionic emissions into a larger radiant energy output.

FIG. 54) Describes the invention's devices using cobalt magnets and mechanical energy together with high voltage ionized argon gas to provide an output of electric energy.

FIG. 55) Describes the inventions devices using cobalt 60 magnets.

FIG. 56) Describes the invention's devices using rare earth elements to increase the power output.

FIG. 57) Describes the invention's devices converting electrical input into mechanical output.

FIG. 58) Describes the invention's “Artificial Liver and Ammonia Diffuser” for cirrosis treatments with blood clotting restorability; comprising of a (1) container having an input tube (14) containing Human (14) Blood with toxins that is filtered with a calcium filter (2) that circulates and moves the blood/fluid with an electric pump (13) and mixes plasma fluid input (3) and connects (13) into and restores blood clotting, a settling tank (5) that connects (13) into another calcium filter (2) tank (6) that then send the filtered blood (13) that has its clottability restored; into another tank (8) wherein the blood is again filtered (2) and slightly heated (7) in the unit (8) tank where the blood is again filtered (2) and slightly heated (7) in the unit (8) tank; wherein the slight heating and calcium mixes with the Human blood and causes ammonia toxins (9) to diffuse into a vent (9) and the blood is sent through a connecting pipe (13) into a cooling unit (10) and filters (10a) down to remove any extra calcium and cools the blood and fluid and sends (13) the cleaned of toxins blood/fluid into a storage tank (11) and a output tube (12) for use if a Human Being and final testing to insure toxin material removal, before being used to the person in need.

1) I the Reverend Daniel Robert Izzo, hereby claim the, “A Resurrection Burial Tomb” that is a novel means to revive a dead person's body from the grave; and providing a 1 little more security from an impending death; will preserve and prevent the human body from decay and will prolong the human life and tissue and return it into a conscious state, together with the means do do the same, and a means to produce energy, to do the same; comprising of:
an artificial womb and a container with fluid having a crystal radio crown and antenna that connects to a radio and energy devices; wherein the deceased person is prevented from decay within said container and where energy is obtained from the radio crown that oscillates into the desceased person's nerves and brain and the entire contianer is within a building and tomb that is weather tight, wherein a nuclear device is used to generate electric energy and heat energy; wherein part of the dead person's bone is converted into a microprocessor and computer; wherein the computer and energy sources, drive a heart lung machine that keeps the dead person's remaining cell tissue alive, together with an artificial heart assistor pump; wherein the deceased person's bone will regain consciousness, using the material of their bone and artificial and organic means cosisting of human bone material; that is convertable into a computer and microprocessor, organically grown body parts; artificailly grown body parts; nerves a muscles; wherein the microprocessor will oscillate of the dead person, from the said bone material; being used will oscillated of the person's life frequency; together with artifically grown nerves and olfactory nerves that grow, reconstructed nerve patterns of the desceased person; together and cosisting of artificial and organically grown muscle; comprising of silicon and compounds to cause silicon and material mean to expand and contract with energy and force; and organic material; when a voltage of energy is applied; wherein the desceased person's skeleton remains, will be revived to consciousness, using collegen, a microprocessor, coversion to a computer consciouness, artificial muscle that expands and contracts with energy and force, artificial nerves that will allow the dead person's skeleton remants; to independently communicate new consciousness and thoughts; wherein the said microprocessor contains elements of human bone materials that will be able to convert itself into other electric circuit patterns, by inputing the desired pattern stored in its memory; such as imputting radio, for radio circuit, imputting oscilloscope for oscilloscope circuit; wherein said micropressor will recieve and transmitt input data and electric pulses, and will contain information, record data and monitor the burial tomb, comprising of a means to recieve and transmitt data and electric pulses; and contain information, record data and monitor, wherein said microprocessor comprises of: a sheet of flexible insulating plastic paper that contains an elargement of specailly painted and printed computer microchips; integrated electric circuits and having thousands of field effect transistors; capacitors and resistors; that was applied and formed with appliced layers of paint, wherein the sheet of plastic paper is coated with a complete layer of positively doped slicon substrate that is printed and painted in an electric circuit pattern of an insulating coat of silicon dioxide paint; thenan area containg the devices negatively charged electric areas, type-N, will be painted and printed; in a pattern for type-N areas, with a paint that contins silicon and germanium crystals and phosphorus with in the type-N semiconductor paint; wherein then the device's negatively charged electric areas will have extra free electrons and produces N-type, negatively charged electric current conduction; wherein then, another electric circuit pattern is then printed and painted that will conduct the P-type, positively charged electric semiconductor regions with a polysilicon paint that contains silicon, germanium, crystals and boron mixed in the paint; wherein the results of the paint leave gaps in the germanium crystal structure because there is not enough electrons to fill all of the spaces in the crystal and produces a P-type semiconductor patterned region for the device's positive electric current conduction; wherein then another layer of silicon dioxide is printed and painted in an electric circuit insulating pattern and shall have some holes for electric conduction and shall have a printed and painted aluminum or other electric conducting metal paint; applied on it in a circuit pattern and shall provide electric contacts that will recieve electric data pulses of either digital inputs or analog-to-digital converter inputs and the electric contacts will provide the device's digital output or the device's digital-to-analog data output, wherein the microprocessor's input of data will be able to store the data in the device's central processing unit; the device's random access memory; and the device's read only memory and are connected together within the device's address bus; data bus; and control bus and the device's bus system connects to the device's input and output connection; wherein the device can store the data for processing and the device turns data into processed results; into actions and diplays, wherein the enlarging of the pattern of the computer microchip electric circuit; integrated electric circuits, 100+thousands of field effect transistors; capacitors and resistors; and then applying the materials to create the same electric systems; painting and printing upon plastic paper, 100+thousand transistors; capacitors and resistors; on an altered xerography process using the aforementioned materials of plastic, silicon, silicon dioxide, polysilicon, germanium; phospherous; boron, crystals, metal, or painting the electric circuit patterns of the I/C; microprocessor-microchip; 100+thousand transistors, capacitors, resistors, circuits in and enlarged pattern; also by lithographic printing or offset cylinders containing the material of paint and the cicuit designs; or of the layer printing methods that print the materials without shorting the electric circuit, wherein the microprocessor can be altered and produce electric energy, and can be a photoelectric cell on top of the microprocessor and can be altered and have a parrellel along the circuits with arsenic, wherein a photoelectric effect takes place, producing electricity; wherein processes in the Ressurection Burial Tomb, and Conscious Revival System; having interactions therein wherein; prior domestic priorities of the inventor's; cosmic radiowave crown, a Portable Nuclear Powered Engine and Electric Generator; a Hydrualic Leverage Engine, a Microwave Hot Water Boiler Heating System; the Zinc-Carbon Pill and Transistorized Plastic Paper Computer, the Artificial Heart Assister Pump; the Artificial Muscle Robot; and the Thermionic Electron Electric Generator and Artificial Liver Ammonia Diffuser; interact in new and improved functions that preserve the deceased person's and offer a little more security from our deaths.
and electric energy, and power systems; are used as a means to provide the process; wherein a small pump will circulate blood and fluid; that is attached to the person's arteries and veins; wherein when a person's heart fails to pump properly; the automatic device will sense this difunction; and will begin to pump and circulate oxygenated blood and fluid properly throughout the person's body tissues, if any, wherein the pumping system is powered electrically or nuclear energy; wherein the power source to circulate and pump blood fluis is connected to the device; wherein the device is connected to a person's blood ciculatory system and will maintain a fresh supply of oxygen to a deceased person's brain and body; wherein; medication and process of the Zinc-Carbon Pill electrophoresis compounds are used in the process for destroying microbes, fungi, bacteria and vival infection and clove leaf oil topicial paste for herpes virus, and protect the deceased person's tissues infection; wherein an enigine is used to provide the Resussection Burial Tomb; with a power source; wherein the Hydrualic Leverage Engine is used comprising of; a gasolin combustion chamber having gas therein; a diesel compression chamber having diesel gas fiel therein; a hydrualic pheumatic engine having gas therein; and ION engine having gas therein; a heat source within said housings which interacts with gas or fuel, or an energy source to provide output, a piston connected to the output of the energy or heat source; wherein; the piston will act upon a hydraulic jack piston pump and is connected to a lever bar and the lever bar rests upon a fulcrum point and bar, wherein the lever bar rests upon a fulcrum point and bar, wherein the lever bar acts upon a set of weight load pistons that connect to a crankshaft and the crankshaft is connected to a load bearing work wheel; and wherein the output of the energy or heat source shall cause and move the effort pistons to apply a small pressure upon the hydrualic jack piston pump that in turn will apply a larger force of pressure upon the lever bar, that in turn will apply an even larger force of pressure upon the weight load pistons that connects to the crankcase and crankshaft and workwheel; so that the crankshaft, under applied pressure of the effort pistions acting upon the hydraulic jack piston pump and lever bar acting upon the weight load pistons, turns a workwheel with much more force that was initally acted upon it from the energy source or heat source but at less distance, wherein the effort multiplied by the distance from the fulcrum, (effort pistons-lever bar ) equal the weight multiplied by its distance from the fulcrum (weight load pistons), wherein the Ressurection Burial Tomb has a workable engine for the interaction process; wherein the Zinc-Carbon Pill will prevent decay of the human person, wherein the process will electrocute viruses and other microbes within a human host and will explode viruses, bacteria, fungi and other microbes, like an overloaded electric light bulb, without destroying many human cells, with the ability to increase the natural electric current and voltage of a human being, with electro-chemical compounds and a process of electrophoresis; and electrocution and electrostatic activity; comprising of; elements of; zinc and its zinc oxides; zinc sulfides and zinc acetates and other zinc elements, carbon and carbon elements and can be in the form of carbonyl and its group, carboxyl and its group, carboxyl and its group and other carbon based elements, magnesium and its compounds and group, wherein the combining of these elements increases the electrical energy of a Human Being with chemical energy and can shatter and explode viruses, bacteria, fungus and other microbes and parasites that cause diseases and cell death of a Human Being, and can be applied also externally to a Human Host with the same effect together with these elements; Zinc; Carbon; Magnesium; Electrophoresis materials; adramycin; antineoplastic; antipan; arsenic; barium and its sulfides; benzyl; benzoic acid; benzoly peroxide; carbamazephine;carbolated campor, carbolic acid; cerumenolytic; daunomycn; hydrogen peroxide; hydrochloride; keratolic acid; magnesium; mithramycn; ozone; phenol red; phenylactetic acid carboxy; phenyl carbinol; pheny ethyl alchohol; phenylactetic acid carboxy; phenyl carbinol; pheny ethyl alcohol; phenyl meruric nitrate; piperdine; permathrin; dimethylcyclopropanecarboxylate; pyrethrin; piperonyl butoxide; sodium; sodium hydrogen-carbonate; sodium perborate; suger; sulfa; sulfamide; penicillin; carben carbenicillin; radio chemicals; ultra-violet rays; electric jolts; negative ions; positive ions and water; wherein combining these elements increases and amplify the electrical energy of a host human inside the tomb chambers, to a safe level, but will explode with electric energy, viruses, bacteria, funguses and other microbes and parasites without damaging many Human cells and is obtained ny chemical energy and other energy forms and the process and treatment with cemicals can be in the form of liquids, solids, powders, pastes, pill gas and electric energy forms as light radiation energy and electro-static energy absorption means to prevent decay of the deceased person's Human cell life,
and wherein the means for a communication device is used from the Citizen Band Free Public Radiowave Telephone System comprising an audio input, microphone, a frequncy programmer number pad to frequency crystal oscillator.
a programmed radio frequency to signal number display, transmit and recieve antennas.
a keyboard to computer input. a speaker, a video monitor, tv monitor, a fax machine/printer, a data input and computer storage data unit, electric plug input/output, telephone line for wire service, cable input/output wire, video/audio camera that is removable, a source of communication antennas for a radiowave reciever and transmitor and propagated radiowaves; wherein; the invention's Citizen Band Free Public Radiowave Telephone system's; audio input with cable wires; input amplifier, radio frequency amplifier, modulated amplifier or frequency amplifier, modulated amplifier or Frequency amplifier master oscillator, radio frequency amplifier, radio frequency amplifier; radio frequency amplifier, crystal oscillator that is programmable; antenna; pre-selection circuit; a frequency changer; intermediate frequency amplifier; frequency detector; low frequency amplifier speaker; antenna for reciever; local oscillator, will interact and function within the Resurrection Burial Tomb processes and radio crown; as a means for monitoring communication, entertainment fun and data;
wherein the invention uses artificial muscles and produces electric current that is stored in the electric storage unit being maintained/operated by the “Artificial Muscle Robot” and its artificial hands having, artificial muscle that is connected to a frame with movable joints and the artificial hand is covered and protected with a material that permits movement, wherein the artificial muscle will expand and contract with force, using electric current by way of spring wires that is within the artificial muscle that is weaved and patterened and layered like a real human hand having sensors for input/output signals through a central joint palm, that has a computer brain with optical sensors and an input/output communication and recieves transmits radio communication and the robot has arms with artificial hands. wherein the robot is powered by a Portable Nuclear Powered Engine and Electric Generator other electric input means that move and perform work of Human hands tasks can perform to maintain and work for the Resurrection Burial Tomb work functions; wherein the Hydraulic Pneumatic Engine and the “Microwave Hot Water Boiler Heating System with a thourium plated magnetron (2) energy systems, using steam from the microwave hot water boiler to drive and help turn the hydrualic pneumatic engine and electric generator wherein the Artificial Muscle Robot assists in the operation of the tomb's hydraulic pneumatic engine and microwave hot water boiler heating energy system. wherein the Lever Engine c covert 1 ton of torque into 4 tons without gear/transmission for the invention's work functions and other electric systems comprising of the Thermionic Electron Electric Generator, having a power source, cathode, cesium, thorium, container with magnetic force inside, and a high voltage anode chamber with argon gas inside for output, wherein the input of 10 watts of electric power is amplified to about 100 watts of electric power output, for energy creation, and the invention's Hydraulic Pneumatic Engine used for work functions and comprising of thePortable Nuclear Powered Engine that mitts less radiation than 3 tv sets and is used for work functions and electric power generation.
wherein; the invention's rebuilding and preseving Human persons who are deceased or suspended or are being revived into normal function, uses the same means of cryogenic presevation, in the U.S. South Pole, Antartican territory and the invention comprises of an “Artificial Womb” coverted from an oxygen petrofusion machine, wherein the Human's cells are preserved/protected and can grown into normal function, wherein a means of a Human bone being cyrstalized and coverted into a computer microprocessor, for an “Artificial Muscle Robot” consciousness and for some normal function.
and a means for a Human person inside a container that is filled with a prevervative medium that prevents decay and who is wearing a cyrstal radio crown to hear the living, awaiting the day of resurrection,
wherein the invention comprises of the Thermionic Electron-Electric Generator that is used to generate the invention's electric power, that comprises of; containment inside a sealed tube containg a source of input power that heats a thorium tungsten nichorme wire that is inside another sealed tube that contains cesium gas that generate electrons, wherein a thoriated platinum cathode obtains emitted electrons and emits more electrons inside a magnetic field having cobalt magnets, wherein an anode collects the elctrons and sends them to a argon gas ionizer chamber having argon gas therein wherein high voltage electric is present that ionizes the radiation of the argon gas and causes electrons to rush to one electron and positively charged ions to the other electrode and positively charged ions to the other electrode; wherein the electrons join other electrons emitted from the anode at the negative electrode as the radiation and high voltage causes an electric current within the device and sends it to the increased electric output to the invention's electric current supply wherein the invention comprises of an electric generator having radioactive cobalt 60 magnets, and depicts radioactive cobalt 60 magnets that cause electrons to move faster than iron magnets and produces more electric output per calorie than iron magnets and its output is increased by high voltage argon gas ionization inside the electric generator, wherein radioactive cobalt field structure magnets create a magnetic field; wherein coils of wire are mounted so that they can be spun around in a magnetic field with mechanical energy input; wherein the entire device is inside a vacuum tube that is filled with a gas and charged to a very high voltage wherein the radioactive cobalt 60 ionizes the gas spading the elctrically neutral gas into positively charged ions and negatively charged electrons wherein; the positive ions rush to one electrode and the electrons rush to the other electrode wire, wherein this triggers an electric current from one electrode to the other, while the mechanical energy input turns the wire coil electrode and creates electrical output between the cobalt 60 magnetic field magnets wherein magnetic force and ionized gas of radioactive cobalt 60 magnets make electrons move in the wire coil generating an electric current output, and a means for the invention's electric systems connected to the invention's electric wiring that provides amplified electricity; wherein the Thermionic Electron Electric Generator are a source of amplified electric power to the invention's wiring
wherein the invention comprises of devices that convert heat energy into electric current
wherein the invention comprises of a Microwave Boiler, converting thorium/uranium electrons into microwave energy said magnetron microwave device, said microwave magnetron
and a klystron thats cathodes and andoes are plated with thorium and uranium and whose magnets are of radioactive cobalt., wherein the invention's microwave emitting device shall be improved by plating the magnetron's electric cathode with thorium and uranium that is radioactive, the anode is of magnatizable metal, radioactive cobalt, nickel or iron and the anode is plated with radioactive metal; wherein the operation of the invention's magmetron, a high D-C potential is applied between cathode and anode, setting up a radial electric field, wherein the combined forces cause electrons to take a spiral path and when the magnetron, the electric oscillations of the magnetron resonators set up an A-C electric field across the resonator gaps, wherein the thorium and uranium metal cathode, magnetic metal anode of cobalt and plated with radioactive metal, in the magnetron sets up an increase in spiraling reactions when an electric current is applied and causes the magnetron output current to be many times the electric input of the invention's magnetron, and the invention, comprises of electric devices converting thorium uranium, cesium tungsten, nichrome, and argon gas atoms interacting and generating electron emissions into electric energy, and using mechanical energy input and obtaing a greater energy output of electric energy, the invention's, Thermionic Electron-Electric Generator, that coverts a smaller energy power input into a larger electric energy output wherein;
the invention's devices converting heat energy and electro magnetic energy into a larger electric energy output, wherein the invention's devices converting a smaller electric energy input into a larger heating calorie output for room tomb air radiant heat,
wherein the invention's devices converting thermionic emissions into a larger radiant energy output, wherein; the invention's devices using cobalt magnets and mechanical energy together with high voltage ionized argon gas to provide an output of electric energy. wherein; the invention's devices using cobalt 60 magnets, wherein the invention's devices using rare earth elements to increase the power output, wherein the invention's devices converting electric input into mechanical output, wherein the invention's “Artificial Liver and Ammonia Diffuser” for cirrosis treatments with blood clotting restorability; comprising of a container having an input tube containing Human Blood with toxins that is filtered with a calcium filter (2) that circulates and moves the blood/fluid with an electric pump and mixes plasma fluid input and connects into and restores blood clotting; a settling tank that connects into another calcium filter tank that then sends the filtered blood that has its clottability restored; into another tank wherein the blood is again filtered (2) and slightly heated in the unit tank where the blood is again filtered and slightly heated in the unit tank; wherein the slight heating and calcium mixes with the Human blood and causes ammonia toxins to diffuse into a vent and the blood is sent through a connecting pipe into a cooling unit and filters down to remove any extra calcium and cools the blood and fluid and sends the cleaned of toxins blood/fluid into a storage tank and a output tube for use if a Human Being and final testing to insure toxin material removal, before being used to the person in need, and said processes interact within the invention for an outcome; wherein the invention comprises of a “Portable Nuclear Powered and Electric Generator” that comprises of a Stirling type engine; wherein the invention's engine having a doubled walled capsule cylinder that is filled with helium gas and contains a floating piston at the top of the floating piston is a pellet of radioactive material that heats the helium to about 1,200 degrees F., where this very hot helium expands rapidly, then forcefully drives down the piston to the bottom of the cylinder where the tempature is almost 1,100 degrees F., lower and as the piston moves down it forces some of the gas out through a valve in the cylinder floor near the spring bellow and the floating piston acts and pushes against the upper hydraulic jack piston and the upper hydraulic jack piston will push against the helium resvoir and will compress the helium gas and pushes against the helium resvoir and will compress the hydraulic fluid resevoir tat will force the hydraulic jack plumps lower hydraulic pistons that will force and act upon a larger load than was or b acted upon it, and will push this force against the crankcase that will turn and urn the load bearing work wheel and the invention's oil pan is filled with oil and the work wheel will turn an electric generator and the entire device is contained inside of a magnetic bottle that has a coil of a magnetic field metal and the magnetic bottle is contained in a ceramic box that is covered with lead plates an a battery is used to hold the electric current in storage for use of the electric systems of the invention, to provide an electric source for the invention; wherein;
wherein the invention's “Portable Nuclear Powered Engine and Electric Generator” shall also comprise of and consisting of a Tidal regenerator engine that has a double walled capsule cylinder, containg a signal computer that si an electromagnet (29) that is activated to attract another magnet and the movement expands a bellows (22a) that forces a little water up a tube into a water boiler where heat from a radioactive source and a thorium plated magnetron maser protected by a window of quartz will vaporize water into steam and the steam is heated further inside of a super heater tube and the steam then pushes another bellows that, in turn, sends a pulse of hydraulic fluid through the hydraulic jack piston pump and will compress the hydraulic piston that will force and act upon a larger load than was originally acted upon it, and also force a lever and together will push this force agaist the crankshaft that will turn and turn the load bearing work wheel and the engine's oil pan is filled with oil and the work wheel will turn an electric generator and the entire engine is contained in a ceramic box that is covered with lead plates and barium plates and the ceramic box is located within a cement molded building and shall contain barium radiation absorbers and power extinguishers that shall discharge and absorb radiation when the extinguishers sense radiation leakage, wherein electric and power is produced for the invention; wherein;
the invention's, comprises of a :cylinder housing operating inside of a magnetic bottle that is contained in a ceramic box that is covered with lead plates and barium plated and is located within a cement premolded building that contains the barium radiation absorbers and powder extinguishers,
wherein the invention's “Portable Nuclear Powered Engine and Electric Generator” cylindrically shaped housing having water and a source of electric power to be amplified and converted into microwave energy and a source of generating microwave energy having its magnetron cathode tubes plated with radioactive nuclear materials as a source of amplifying the cathode rays that will be coverted into microwave energy to provide an output and a computer that uses either cable wire and or transmits data by radio waves and television KHZ-MHZ-GHZ waves to a radio and television reciever that is connected to a computer that is connected to a magnetic video/audio tape recorder for recording computer data inexpensively, wherein 1 vhs magnetic tape can hold 1.5 gigabytes of computer data,
wherein the invention's “Portable Nuclear Powered Engine and Electric Generator”, cylindrically shaped housing and thorium plated magnetron that emits microwave energy and a magnet recieving cosmic radiowaves and converting the energy into usable electric current by using a crystal radioset with an antenna sensitive at collecting radiowave energy and using and storing it inside of a battery and the cosmic radiowaves shall also be recieved and recorded on magnetic tape and stored for anaylsis of fluctuations and data pulses inside of the unit's audio-video recorder and accessed by the computer key code

2) A Thermionic Elecron Electric Generator and Artificial Ammonia Diffuser as defined in claim 1 and in the specification, in which very low energy input creates a higher energy output.

3) Microwave Hot Water Boiler Heating System as defined in claim 1 and in the specification, in which low energy input creates a higher energy output.

4) A Portable Nuclear Powered Engine and Electric Generator as defined in claim 1 and in the specification, in which low level nuclear energy input creates a steady means of power, for charging batteries and providing electric current.

5) A Hydrualic Leverage Engine as defined in claim 1 and the specification; wherein 1 unit of input work is converted into 4 units of output work using a lever and hyraulic jacks.

6) The Zinc-Carbon Pill and Transistorized Plastic Paper Computer as defined in claim 1 and the specification, wherein microbes, viruses, bacteria and fungi are electrocuted without much human cell destruction, and information data is stored.

7) The Artificial Muscle Robot as defined in claim 1 and the specification wherein, the transistorized plastic paper computer is used a the cpu and the power souce is a portable nuclear powered engine and electric generator.

8) The Artificial Heart Assistor Pump as defined in claim 1 and the specification wherein a energy source is used to provide an output.

9) The Citizen's Band Free Public Radiowave Telephone as defined in claim 1 and the specification, wherein the transistorized plastic paper computer is used for radio propagation.

10) A Resurrection Machine and Consciouness Revival System as defined in claim 1 and the specification is incorporated with the Resurrection Burial Tomb, to provide output.
